(19) United States
(12) Patent Application Publication
(10) Pub. No.: US 2010/0047266 A1
(43) Pub. Date: Feb. 25, 2010

(54) CHIMERIC VIRUS-LIKE PARTICLES

(75) Inventor: Joel R. Haynes, Bozeman, MT (US)

Correspondence Address: MORRISON & FOERSTER LLP, 425 MARKET STREET, SAN FRANCISCO, CA, 94105-2482, US

(73) Assignee: LigoCyte Pharmaceuticals, Inc., Bozeman, MT (US)

(21) Appl. No.: 12/375,281

(22) PCT Filed: Jul. 27, 2007
(86) PCT No.: PCT/US07/16938

§ 371 Date: Oct. 12, 2009

Related U.S. Application Data
(60) Provisional application No. 60/833,944, filed on Jul. 27, 2006.

(51) Int. Cl. A61K 39/12; C07K 14/005; C12N 9/96; C07H 21/04

(52) U.S. Cl. 424/186.1; 530/350; 435/188; 530/395; 536/23.1; 435/69.1

(57) ABSTRACT

Chimeric virus-like particles including gag polypeptides are described. Virus-like particles are generated with a gag polypeptide and lipid raft-associated polypeptide linked to an antigen that is not naturally associated with a lipid raft. Preferred methods of generation include expression in insect cells.

### GOVERNMENT SUPPORT

This invention was made with United States Government support under Grant No. W81XWH-05-C-0135 and Grant No. W81XWH-05-C-0150 from the U.S. Army Medical Research and Material Command. The United States Government has certain rights in this invention.

### FIELD OF THE INVENTION

The present invention relates to the field of virus-like particles. In particular, chimeric virus-like particles having a gag polypeptide and a lipid raft-associated polypeptide linked to an antigen not naturally associated with a lipid raft are disclosed herein.

### BACKGROUND OF THE INVENTION

Virus-like particles (VLPs) offer several advantages over conventional vaccine technology. An important advantage of VLPs for vaccine development is that they mimic native viruses in terms of three-dimensional structure and the ability to induce neutralizing antibody responses to both primary and conformational epitopes and therefore should prove more immunogenic than other vaccine formulations. Unlike viral vectored approaches, VLPs exhibit no problem with pre-existing immunity, thus allowing for recurrent use.

Many traditional vaccines are parenterally administered and largely induce (or boost) systemic IgG responses protecting the lower respiratory tract. New vaccine approaches that induce mucosal responses are more desirable as they can restrict virus growth in both the upper and lower respiratory tracts and are likely the best vaccine approach for individual protection and reduction of transmission (15). In addition to providing protection in both the upper and lower respiratory tracts, intranasal vaccines avoid the complications of needle inoculations and provide a means of inducing both mucosal and systemic humoral and cellular responses via interaction of particulate and/or soluble antigens with nasopharyngeal-associated lymphoid tissues (NALT) (16-19). VLPs in general appear well suited for the induction of mucosal and systemic immunity following intranasal delivery as has been shown for rotavirus, norovirus, and papilloma virus VLPs (28-31).

Despite the advantages of VLPs, development of enveloped-VLPs (VLPs derived from enveloped viruses containing integral membrane proteins) as vaccines is currently limited by several problems. One of the most significant problems is the limited range of antigens and thus diseases for which enveloped-VLP vaccines can be developed. Since incorporation of antigens into VLPs appears to require association with the lipid raft domains where the viral capsid proteins initiate assembly of viral particles, current methods for VLP production are limited to the use of viral capsid protein to form VLPs containing native viral antigens which naturally associate with the lipid raft. Thus, there is a need for a VLP platform technology that allows production of VLPs containing any type of antigen, including those viral antigens which do not naturally associate with a lipid raft, antigens from viruses other than the source of the capsid protein, antigens from other pathogens, such as bacteria, fungus, protozoa, helminth, yeast, etc., tumor antigens, as well as allergens.

Another significant problem with existing VLP technology is the inability to produce sufficient yields of VLPs. For example, although influenza matrix-derived VLPs are immunogenic, their poor yield makes them a poor choice to date for an alternate form of influenza vaccine. Thus, there is also a need for a VLP vaccine platform technology that can generate sufficient quantities of VLPs for vaccine production.

### SUMMARY

The present invention meets these needs by providing various methods and compositions as disclosed herein for production and use of chimeric VLPs containing antigens that do not associate naturally with a lipid raft from all types of pathogens and which may also be generated in sufficient quantities for vaccine production.

In one aspect, the invention provides chimeric virus-like particles having a gag polypeptide and a lipid raft-associated polypeptide linked to an antigen, where the antigen is not naturally associated with a lipid raft. The linkage may be a covalent bond, an ionic interaction, a hydrogen bond, an ionic bond, a van der Waals force, a metal-ligand interaction, or an antibody-antigen interaction. The covalent bond can be a peptide bond, a carbon-oxygen bond, a carbon-sulfur bond, a carbon-nitrogen bond, a carbon-carbon bond or a disulfide bond. The gag polypeptide is preferably from a retrovirus which may include murine leukemia virus, human immunodeficiency virus, Alpharetroviruses, Betaretroviruses, Gammaretroviruses, Deltaretroviruses, Deltaretroviruses and Lentiviruses.

The lipid raft-associated polypeptide can be any polypeptide that is either directly or indirectly associated with a lipid raft, for example, it may be an integral membrane protein. In preferred embodiments, the lipid raft-associated polypeptide is a hemagglutinin polypeptide, a neuraminidase polypeptide, a fusion protein polypeptide, a glycoprotein polypeptide, or an envelope protein polypeptide.

The antigen can be any substance capable of eliciting an immune response, such as a protein, a polypeptide, a glycopolypeptide, a lipopolypeptide, a peptide, a polysaccharide, a polysaccharide conjugate, a peptide or non-peptide mimic of a polysaccharide, a small molecule, a lipid, a glycolipid, or a carbohydrate. Preferably, the antigen is a viral antigen, a bacterial antigen, a eukaryotic pathogen antigen, a tumor antigen or an allergen.

In another aspect, the virus-like particles described herein also includes an adjuvant associated with the virus-like particle. The adjuvant may be located inside the VLP, preferably by being covalently linked to the gag polypeptide. In other embodiments, the adjuvant is located outside the virus-like particle, preferably by being covalently linked to the lipid raft-associated polypeptide. Preferred examples of polypeptide adjuvants include flagellin and adjuvant-active fragments thereof, cytokines, colony-stimulating factors (e.g., GM-CSF, CSF, and the like); interferons; tumor necrosis factor; interleukin-2, -7, -12, and other like growth factors.

In yet another aspect, the invention provides virus-like particle expression vector systems having a first nucleotide sequence encoding a gag polypeptide and a second nucleotide sequence encoding a lipid raft-associated polypeptide linked to an antigen, where the antigen is not naturally associated with a lipid raft, and wherein upon expression in a cellular host, the polypeptides form a virus-like particle. In one embodiment, the first and second nucleotide sequences are in a single expression vector and preferably operably linked to separate promoters, but may be linked to a single promoter. In another embodiment, the first and second nucleotide sequences are in multiple expression vectors.

In still yet another aspect, the invention provides methods for producing a virus-like particle by providing one or more expression vectors, together which express a gag polypeptide and a lipid raft-associated polypeptide linked to an antigen, where the antigen is not naturally associated with a lipid raft; introducing the one or more expression vectors into a cell; and expressing the gag polypeptide and the lipid raft-associated polypeptide linked to an antigen to produce the virus-like particle. In preferred embodiments, one or more expression vectors is a viral vector. The viral vector may be a baculovirus, an adenovirus, a herpesvirus, a poxvirus, or a retrovirus. The cell may be an insect cell or a mammalian cell. In some embodiments, the methods also include the step of recovering the virus-like particle from the media in which the cell is cultured.

In another aspect, the invention provides methods for treating or preventing a disease or symptom of the immune system by administering an immunogenic amount of any of the chimeric influenza virus-like particles describe herein. In certain embodiments, the administering induces a protective immunization response in the subject. In certain embodiments, the administering is by subcutaneous delivery, transcutaneous delivery, intradermal delivery, subdermal delivery, intramuscularly delivery, peroral delivery, oral delivery, intranasal delivery, buccal delivery, sublinqual delivery, intraperitoneal delivery, intravaginal delivery, anal delivery or intracranial delivery.

Another aspect of the chimeric influenza virus-like particles disclosed herein is pharmaceutical compositions which can include an immunogenic or therapeutic amount of any of the chimeric virus-like particles describe herein. Such pharmaceutical compositions preferably will include a pharmaceutically acceptable carrier that is preferably formulated for the preferred delivery method.

In another aspect, the invention provides pharmaceutical compositions that include the VLPs as disclosed herein. In preferred embodiments the pharmaceutical compositions will include a pharmaceutically acceptable carrier.

In another aspect, the invention provides a chimeric virus-like particle that includes a gag polypeptide and a non-viral lipid raft-associated polypeptide. Such VLPs include all of the various embodiments of the other VLPs disclosed herein. In certain embodiments, the lipid raft-associated polypeptide may a GPI anchor polypeptide, a myristoylation sequence polypeptide, a palmitoylation sequence polypeptide, a double acetylation sequence polypeptide, a signal transduction polypeptide, or a membrane trafficking polypeptide or preferably a cavelin polypeptide, a flotillin polypeptide, a syntaxin-1 polypeptide, a syntaxin-4 polypeptide, a synapsin I polypeptide, an adducin polypeptide, a VAMP2 polypeptide, a VAMP/synaptobrevin polypeptide, a synaptobrevin II polypeptide, a SNARE polypeptide, a SNAP-25 polypeptide, a SNAP-23 polypeptide, a synaptotagmin I polypeptide, or a synaptotagmin II polypeptide. Such chimeric virus-like particles that include a gag polypeptide and a non-viral lipid raft-associated polypeptide also include all of the aforementioned aspects and embodiments throughout the disclosure including, without limitation, expression vector systems, methods of production, methods of treatment and prevention, and pharmaceutical compositions.

### SUMMARY OF THE FIGURES

FIG. 1 shows western blots of the media from Sf9 cells infected with separate Gag, HA or control vectors and with HA-gag-NA triple vectors. (A) was probed with anti-Gag antibodies and (B) was probed with anti-HA antibodies.

FIG. 2 shows western blots of fractions from a sucrose step gradient recentrifugation of pelleted HA-gag-NA VLPs. (A) was probed with anti-Gag antibodies and (B) was probed with anti-HA antibodies.

FIG. 3 shows the arrangement of coding sequences in the triple expression vector for Example 1, below.

FIG. 4 shows arrangement of coding elements in the triple baculovirus expression vector encoding the PA-modified HA along with Gag and NA.

FIG. 5 shows the arrangement of coding elements in the double baculovirus expression vector encoding RSV F protein and Gag.

### DETAILED DESCRIPTION OF THE PREFERRED EMBODIMENTS

The present invention includes gag polypeptides as the basis for formation of chimeric VLPs. A preferred method of generating the VLPs is by expression in insect cells, preferably including coexpression of polypeptide antigens, because of the significant yields of gag VLPs that can be obtained from a variety of retroviruses in the baculovirus expression system (23, 24, 46, 49, 52-58). Gag polypeptides inherently include C-terminal extensions in the natural retroviral assembly process in that functional gag proteins naturally have large C-terminal extensions containing retroviral protease, reverse transcriptase, and integrase activity due to ribosomal frameshifting. Production of functional gag proteins with artificial extensions has been accomplished for both RSV gag (59) and MLV gag (60). This flexibility in manipulation of the gag C-terminus provides an important site for inclusion of other polypeptides such as other antigens and immunostimulatory protein sequences.

The production of chimeric VLPs containing a core particle from one virus and surface antigens from another is called pseudotyping. Gag polypeptides have been efficiently pseudotyped with influenza HA and NA, presumably since these proteins are concentrated within lipid raft domains (61, 62) while myristolated gag proteins also concentrate at the inner surface of lipid raft domains during the budding process (63).

The present invention described herein further includes lipid-raft associated polypeptides linked to an antigen which is not naturally associated with a lipid raft as a basis for formation of chimeric VLPs. Without wishing to be bound by any theory, it is believed that by virtue of association of the antigen with the lipid-raft associated polypeptide, a chimeric VLP containing the antigen will be formed.

The practice of the disclosed methods and protocols will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### DEFINITIONS

The “Gag polypeptide” as used herein is the retrovirus-derived structural polypeptide that is responsible for formation of the virus-like particles described herein. In some embodiments, the gag polypeptide may be purposely mutated in order to affect certain characteristics such as the propensity to package RNA or the efficiency of particle formation and budding. The genome of retroviruses codes for three major gene products: the gag gene coding for structural proteins, the pol gene coding for reverse transcriptase and associated proteolytic polypeptides, nuclease and integrase associated functions, and env whose encoded glycoprotein membrane proteins are detected on the surface of infected cells and also on the surface of mature released virus particles. The gag genes of all retroviruses have an overall structural similarity and within each group of retroviruses are conserved at the amino acid level. The gag gene gives rise to the core proteins excluding the reverse transcriptase.

For MLV the Gag precursor polyprotein is Pr65^{Gag} and is cleaved into four proteins whose order on the precursor is NH₂-p15-pp12-p30-p10-COOH. These cleavages are mediated by a viral protease and may occur before or after viral release depending upon the virus. The MLV Gag protein exists in a glycosylated and a non-glycosylated form. The glycosylated forms are cleaved from gPr80^{Gag} which is synthesized from a different inframe initiation codon located upstream from the AUG codon for the non-glycosylated Pr65^{Gag}. Deletion mutants of MLV that do not synthesize the glycosylated Gag are still infectious and the non-glycosylated Gag can still form virus-like particles, thus raising the question over the importance of the glycosylation events. The post translational cleavage of the HIV-1 Gag precursor of pr55^{Gag} by the virus coded protease yields the N-myristoylated and internally phosphorylated p17 matrix protein (p17MA), the phosphorylated p24 capsid protein (p24CA), and the nucleocapsid protein p15 (p15NC), which is further cleaved into p9 and p6.

Structurally, the prototypical Gag polyprotein is divided into three main proteins that always occur in the same order in retroviral gag genes: the matrix protein (MA) (not to be confused with influenza matrix protein M1, which shares the name matrix but is a distinct protein from MA), the capsid protein (CA), and the nucleocapsid protein (NC). Processing of the Gag polyprotein into the mature proteins is catalyzed by the retroviral encoded protease and occurs as the newly budded viral particles mature. Functionally, the Gag polyprotein is divided into three domains: the membrane binding domain, which targets the Gag polyprotein to the cellular membrane; the interaction domain which promotes Gag polymerization; and the late domain which facilitates release of nascent virions from the host cell. The form of the Gag protein that mediates assembly is the polyprotein. Thus, the assembly domains need not lie neatly within any of the cleavage products that form later. The Gag polypeptide as included herein therefore includes the important functional elements for formation and release of the VLPs. The state of the art is quite advanced regarding these important functional elements. See, e.g., Hansen et al. J. Virol 64, 5306-5316, 1990; Will et al., AIDS 5, 639-654, 1991; Wang et al. J. Virol. 72, 7950-7959, 1998; McDonnell et al., J. Mol. Biol. 279, 921-928, 1998; Schultz and Rein, J. Virol. 63, 2370-2372, 1989; Accola et al., J. Virol. 72, 2072-2078, 1998; Borsetti et al., J. Virol., 72, 9313-9317, 1998; Bowzard et al., J. Virol. 72, 9034-9044, 1998; Krishna et al., J. Virol. 72, 564-577, 1998; Wills et al., J. Virol. 68, 6605-6618, 1994; Xiang et al., J. Virol. 70, 5695-5700, 1996; Garnier et al., J. Virol. 73, 2309-2320, 1999.

As used in the VLPs of the present invention, the gag polypeptide shall at a minimum include the functional elements for formation of the VLP. The gag polypeptide may optionally include one or more additional polypeptides that may be generated by splicing the coding sequence for the one or more additional polypeptides into the gag polypeptide coding sequence. A preferred site for insertion of additional polypeptides into the gag polypeptide is the C-terminus.

Preferred retroviral sources for Gag polypeptides include murine leukemia virus, human immunodeficiency virus, Alpharetroviruses (such as the avian leucosis virus or the Rous sarcoma virus), Betaretroviruses (such as mouse mammary tumor virus, Jaagsiekte sheep retrovirus and Mason-Phizer monkey virus), Gammaretroviruses (such as murine leukemia virus, feline leukemia virus, reticuloendotheliosis virus and gibbon ape leukemia virus), Deltaretroviruses (such as human T-lymphotrophic virus and bovine leukemia virus), Epsilonretroviruses (such as walleye dermal sarcoma virus), or Lentiviruses (human immunodeficiency virus type 1, HIV-2, simian immunodeficiency virus, feline immunodeficiency virus, equine infectious anemia virus, and caprine arthritis encephalitis virus).

The “lipid raft” as used herein refers to the cell membrane microdomain in which the gag polypeptide concentrates during the viral particle assembly process.

A “lipid raft-associated polypeptide” as used herein refers to any polypeptide that is directly or indirectly associated with a lipid raft. The particular lipid raft-associated polypeptide used in the invention will depend on the desired use of the chimeric virus-like particle.

The lipid raft-associated polypeptide can be an integral membrane protein, a protein directly associated with the lipid raft via a protein modification which causes association with the membrane, or a polypeptide with an indirect association with the lipid raft via a lipid raft-associated polypeptide.

Many proteins with lipid anchors associate with lipid rafts. Lipid anchors that couple polypeptides to lipid rafts include GPI anchors, myristoylation, palmitoylation, and double acetylation.

Many different types of polypeptides are associated with lipid rafts. Lipid rafts function as platforms for numerous biological activities including signal transduction, membrane trafficking, viral entry, viral assembly, and budding of assembled particles and are therefore associated with the various polypeptides involved in these processes.

The various types of polypeptides involved in signaling cascades are associated with lipid rafts that function as signaling platforms. One type of lipid raft which functions as signaling platform is called a caveolae. It is a flask shaped invagination of the plasma-membrane which contains polypeptides from the caveolin family (e.g., caveolin and/or flottillin).

Membrane trafficking polypeptides are associated with lipid rafts which function as membrane trafficking platforms. Examples include the proteins involved in endocytosis and excocytosis, such as syntaxin-1, syntaxin-4, synapsin I, adducin, VAMP2, VAMP/synaptobrevin, synaptobrevin II, SNARE proteins, SNAP-25, SNAP-23, synaptotagmin I, synaptotagmin II, and the like.

Viral receptors, receptor-coreceptor complexes, any other components which help modulate the entry process are associated with lipid rafts which function as specialized membrane trafficking platforms for viral entry. Examples of lipid raft-associated viral receptors include the decay accelerating factor (DAF or CD55), a GPI-anchored membrane glycoprotein that is a receptor for many enteroviruses; the receptor for group A rotaviruses, a complex containing multiple components including gangliosides, Hsc70 protein, alpha2-beta1 and alpha5-beta2 integrins; glycoproteins of several enveloped viruses like HIV, MLV, measles, and Ebola; and polypeptides involved in HIV entry like CD5, CCR5, and nef. See Chazal and Gerlier, 2003, Virus Entry, Assembly, Budding, and Membrane Rafts, Microbiol. & Mol. Bio. Rev. 67(2):226-237.

Polypeptides involved in viral particle assembly are associated with lipid rafts functioning as viral assembly platforms. Examples of such polypeptides include the HA and NA influenza envelope glycoproteins, the H and mature F1-F2 fusion proteins from measles, and the gp160, gp41, and Pr55gag from HIV. See Chazal and Gerlier, 2003, Virus Entry, Assembly, Budding, and Membrane Rafts, Microbiol. And Mol. Bio. Rev. 67(2):226-237.

Polypeptides involved in budding of assembled virus are associated with lipid rafts that function as viral budding platforms. There is data suggesting that HIV-1 budding from the host cell occurs in membrane rafts. See Chazal and Gerlier, 2003, Virus Entry, Assembly, Budding, and Membrane Rafts, Microbiol. And Mol. Bio. Rev. 67(2):226-237. General information about polypeptides involved in viral budding can be found in Fields Virology (4th ed.) 2001.

Preferred lipid-raft associated polypeptides include viral polypeptides such as hemagglutinin polypeptide, neuraminidase polypeptide, fusion protein polypeptide, glycoprotein polypeptide, and envelope protein polypeptide. Each of these polypeptide can be from any type of virus; however, certain embodiments include envelope protein from HIV-1 virus, fusion protein from respiratory syncytial virus or measles virus, glycoprotein from respiratory syncytial virus, herpes simplex virus, or Ebola virus, and hemagglutinin protein from measles virus.

Preferred non-viral pathogen lipid-raft associated polypeptides may be obtained from pathogenic protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like. Such non-viral lipid-raft associated polypeptides may be used without being liked to an antigen not naturally associated with a lipid-raft as the lipid raft-associated polypeptide itself will act as the antigen.

A preferred example of a viral lipid-raft associated polypeptide is a hemagglutinin polypeptide. The “hemagglutinin polypeptide” as used herein is derived from the influenza virus protein that mediates binding of the virus to the cell to be infected. Hemagglutinin polypeptides may also be derived from the comparable measles virus protein. The protein is an antigenic glycoprotein found anchored to the surface of influenza viruses by a single membrane spanning domain. At least sixteen subtypes of the influenza hemagglutinin have been identified labeled H1 through H16. H1, H2, and H3, are found in human influenza viruses. Highly pathogenic avian flu viruses with H5 or H7 hemagglutinins have been found to infect humans at a low rate. It has been reported that single amino acid changes in the avian virus strain's type H5 hemagglutinin have been found in human patients that alters the receptor specificity to allow the H5 hemagglutinin to significantly alter receptor specificity of avian H5N1 viruses, providing them with an ability to bind to human receptors (109 and 110). This finding explains how an H5N1 virus that normally does not infect humans can mutate and become able to efficiently infect human cells.

Hemagglutinin is a homotrimeric integral membrane polypeptide. The membrane spanning domain naturally associates with the raft-lipid domains, which allows it to associate with the gag polypeptides for incorporation into VLPs. It is shaped like a cylinder, and is approximately 135 Å long. The three identical monomers that constitute HA form a central coiled-coil and a spherical head that contains the sialic acid binding sites, which is exposed on the surface of the VLPs. HA monomers are synthesized as a single polypeptide precursor that is glycosylated and cleaved into two smaller polypeptides: the HA1 and HA2 subunits. The HA2 subunits form the trimeric coiled-coil that is anchored to the membrane and the HA1 subunits form the spherical head.

As used in the VLPs of the present invention, the hemagglutinin polypeptide shall at a minimum include the membrane anchor domain. The hemagglutinin polypeptide may be derived from any influenza virus type, subtype, strain or substrain, preferable from the H1, H2, H3, H5, H7, and H9 hemagglutinins. In addition, the hemagglutinin polypeptide may be a chimera of different influenza hemagglutinins. The hemagglutinin polypeptide preferably includes one or more additional antigens not naturally associated with a lipid raft that may be generated by splicing the coding sequence for the one or more additional polypeptides into the hemagglutinin polypeptide coding sequence. A preferred site for insertion of additional polypeptides into the hemagglutinin polypeptide is the N-terminus.

Another preferred example of a viral lipid-raft associated polypeptide is a neuraminidase polypeptide. The “neuraminidase polypeptide” as used herein is derived from the influenza virus protein that mediates release of the influenza virus from the cell by cleavage of terminal sialic acid residues from glycoproteins. The neuraminidase glycoprotein is expressed on the viral surface. The neuraminidase proteins are tetrameric and share a common structure consisting of a globular head with a beta-pinwheel structure, a thin stalk region, and a small hydrophobic region that anchors the protein in the virus membrane by a single membrane spanning domain. The active site for sialic acid residue cleavage includes a pocket on the surface of each subunit formed by fifteen charged amino acids, which are conserved in all influenza A viruses. At least nine subtypes of the influenza neuraminidase have been identified labeled N1 through N9.

As used in the VLPs of the present invention, the neuraminidase polypeptide shall at a minimum include the membrane anchor domain. The state of the art regarding functional regions is quite high. See, e.g., Varghese et al., Nature 303, 35-40, 1983; Colman et al., Nature 303, 41-44, 1983; Lentz et al., Biochem, 26, 5321-5385, 1987; Webster et al., Virol. 135, 30-42, 1984. The neuraminidase polypeptide may be derived from any influenza virus type, subtype strain or substrain, preferably from the N1 and N2 neuraminidases. In addition, the neuraminidase polypeptide may be a chimera of different influenza neuraminidase. The neuraminidase polypeptide preferably includes one or more additional antigens that are not naturally associated with a lipid raft that may be generated by splicing the coding sequence for the one or more additional polypeptides into the hemagglutinin polypeptide. A preferred site for insertion of additional polypeptides into the neuraminidase polypeptide coding sequence is the C-terminus.

Another preferred example of a lipid raft associated peptide is an insect derived adhesion protein termed fasciclin I (FasI). The “fasciclin I polypeptide” as used herein is derived from the insect protein that is involved in embryonic development. This non-viral protein can be expressed in an insect cell baculovirus expression system leading to lipid raft association of FasI (J. Virol. 77, 6265-6273, 2003). It therefore follows that attachment of a heterologous antigen to a fasciclin I polypeptide will lead to incorporation of the chimeric molecule into VLPs when co-expressed with gag. As used in the VLPs of the present invention, the fasciclin I polypeptide shall at a minimum include the membrane anchor domain.

Another preferred example of a lipid raft associated peptide is a viral derived attachment protein from RSV named the G glycoprotein. The “G glycopolypeptide” as used herein is derived from the RSV G glycoprotein. Recent data has demonstrated that lipid raft domains are important for RSV particle budding as they are for influenza virus (Virol 327, 175-185, 2004; Arch. Virol. 149, 199-210, 2004; Virol. 300, 244-254, 2002). The G glycoprotein from RSV is a 32.5 kd integral membrane protein that serves as a viral attachment protein as well as a protective antigen for RSV infection. As with the hemagglutinin from influenza virus, its antigenicity may enhance the antigenicity of any non-lipid raft antigens attached to it. Since RSV does not naturally express a protein with neuraminidase activity, it is likely that VLPs composed of gag and RSV G will not require the presence of NA for efficient production and release. Therefore, development of an expression vector encoding MLV gag and a G glycopolypeptide will result in the production of VLPs containing the G glycopolypeptide integrated into the membrane. Any modifications to the G glycopolypeptide in the way of non-lipid raft foreign antigen attachment will result in chimeric VLPs capable of inducing significant immune responses to the foreign antigen.

The terms “chimeric virus-like particle” and “VLP” are used interchangeably throughout except where VLP by its context is referring to a virus-like particle that is not formed with a gag polypeptide as disclosed herein.

Antigens

The present invention provides gag polypeptides and lipid raft-associated polypeptides as a readily adaptable platform for forming VLPs containing antigens which are not naturally associated with a lipid raft. This section describes preferred antigens for use with the disclosed VLPs.

### Linkage Between Antigen and Lipid Raft-Associated Polypeptide

As a means for forming VLPs containing antigens not naturally associated with a lipid raft, a linkage is formed between the lipid raft-associated polypeptide and the antigen. The lipid-raft associated polypeptide may be linked to a single antigen or to multiple antigens to increase immunogenicity of the VLP, to confer immunogenicity to various pathogens, or to confer immunogenicity to various strains of a particular pathogen.

The linkage between the antigen and a lipid raft-associated polypeptide can be any type of linkage sufficient to result in the antigen being incorporated into the VLP. The bond can be a covalent bond, an ionic interaction, a hydrogen bond, an ionic bond, a van der Waals force, a metal-ligand interaction, or an antibody-antigen interaction. In preferred embodiments, the linkage is a covalent bond, such as a peptide bond, carbon-oxygen bond, a carbon-sulfur bond, a carbon-nitrogen bond, a carbon-carbon bond, or a disulfide bond.

The antigen may be produced recombinantly with an existing linkage to the lipid-raft associated polypeptide or it may be produced as an isolated substance and then linked at a later time to the lipid-raft associated polypeptide.

Antigen Types

The antigens as used herein can be any substance capable of eliciting an immune response and which does not naturally associate with a lipid raft. Antigens include, but are not limited to, proteins, polypeptides (including active proteins and individual polypeptide epitopes within proteins), glycopolypeptides, lipopolypeptides, peptides, polysaccharides, polysaccharide conjugates, peptide and non-peptide mimics of polysaccharides and other molecules, small molecules, lipids, glycolipids, and carbohydrates. If the antigen does not naturally associate either directly or indirectly with a lipid raft, it would not be expected to be incorporated into a VLP without linkage to a lipid raft-associated polypeptide. The antigen can be any antigen implicated in a disease or disorder, e.g., microbial antigens (e.g., viral antigens, bacterial antigens, fungal antigens, protozoan antigens, helminth antigens, yeast antigens, etc.), tumor antigens, allergens and the like.

Sources for Antigens

The antigens described herein may be synthesized chemically or enzymatically, produced recombinantly, isolated from a natural source, or a combination of the foregoing. The antigen may be purified, partially purified, or a crude extract.

Polypeptide antigens may be isolated from natural sources using standard methods of protein purification known in the art, including, but not limited to, liquid chromatography (e.g., high performance liquid chromatography, fast protein liquid chromatography, etc.), size exclusion chromatography, gel electrophoresis (including one-dimensional gel electrophoresis, two-dimensional gel electrophoresis), affinity chromatography, or other purification technique. In many embodiments, the antigen is a purified antigen, e.g., from about 50% to about 75% pure, from about 75% to about 85% pure, from about 85% to about 90% pure, from about 90% to about 95% pure, from about 95% to about 98% pure, from about 98% to about 99% pure, or greater than 99% pure.

One may employ solid phase peptide synthesis techniques, where such techniques are known to those of skill in the art. See Jones, The Chemical Synthesis of Peptides (Clarendon Press, Oxford) (1994). Generally, in such methods a peptide is produced through the sequential addition of activated monomeric units to a solid phase bound growing peptide chain.

Well-established recombinant DNA techniques can be employed for production of polypeptides either in the same vector as the lipid-raft associated polypeptide, where, e.g., an expression construct comprising a nucleotide sequence encoding a polypeptide is introduced into an appropriate host cell (e.g., a eukaryotic host cell grown as a unicellular entity in in vitro cell culture, e.g., a yeast cell, an insect cell, a mammalian cell, etc.) or a prokaryotic cell (e.g., grown in in vitro cell culture), generating a genetically modified host cell; under appropriate culture conditions, the protein is produced by the genetically modified host cell.

Viral Antigens

Suitable viral antigens include those associated with (e.g., synthesized by) viruses of one or more of the following groups: Retroviridae (e.g. human immunodeficiency viruses, such as HIV-1 (also referred to as HTLV-III, LAV or HTLV-III/LAV, or HIV-III); and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis, including Norwalk and related viruses); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus, Metapneumoviridae (e.g., Avian pneumovirus, Human metapneumovirus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 (HHV-1) and 2 (HHV-2), varicella zoster virus (HHV-3), Epstein Barr virus (HHV-4), cytomegalovirus (CMV) (HHV-5)); Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); and astroviruses.

Norvirus Antigens

The VLPs disclosed herein may preferably include various antigens from the Norovirus family. Noroviruses, also called “Norwalk-like viruses” represent one of four genera within the Caliciviridae virus family. Within the Norovirus genus there are two major genetic groups that have been designated Genogroup I and Genogroup II. Genogroup I Norovirus strains include Norwalk virus, Southampton virus, Desert Shield virus, and Chiba virus. Genogroup II Norovirus strains include Houston virus, Hawaii virus, Lordsdale virus, Grimsby virus, Mexico virus, and the Snow Mountain agent (Parker, T. D., et al. J. Virol. (2005) 79(12):7402-9; Hale, A. D., et al. J. Clin. Micro. (2000) 38(4):1656-1660). Norwalk virus (NV) is the prototype strain of a group of human caliciviruses responsible for the majority of epidemic outbreaks of acute viral gastroenteritis worldwide. The Norwalk virus capsid protein has two domains: the shell domain (S) and the protruding domain (P). The P domain (aa 226-530, Norwalk strain numbering) is divided into two subdomains, P1 and P2. The P2 domain is a 127 aa insertion (aa 279-405) in the P1 domain and is located at the most distal surface of the folded monomer. The P2 domain is the least conserved region of VP1 among norovirus strains, and the hypervariable region within P2 is thought to play an important role in receptor binding and immune reactivity. Given the external location of the P domain, it is the preferred antigen or source of polypeptide epitopes for use as antigens for the VLP vaccines disclosed herein. The P2 domain is a preferred antigen for Genogroup I or Genogroup II Norovirus strains. Even more preferred is the mAb 61.21 epitope recently identified as lying in a region of the P2 domain conserved across a range of norovirus strains, as well as the mAb 54.6 epitope (Lochridge, V. P., et al. J. Gen. Virol. (2005) 86:2799-2806).

Influenza Antigens

The VLPs disclosed herein may include various antigens from influenza other than, or in addition to, hemagglutinin and neuraminidase. A preferred additional influenza antigen is the M2 polypeptide. The M2 polypeptide of influenza virus is a small 97 amino acid class III integral membrane protein encoded by RNA segment 7 (matrix segment) following a splicing event (80, 81). Very little M2 exists on virus particles but it can be found more abundantly on infected cells. M2 serves as a proton-selective ion channel that is necessary for viral entry (82, 83). It is minimally immunogenic during infection or conventional vaccination, explaining its conservation, but when presented in an alternative format it is more immunogenic and protective (84-86). This is consistent with observations that passive transfer of an M2 monoclonal antibody in vivo accelerates viral clearance and results in protection (87). When the M2 external domain epitope is linked to HBV core particles as a fusion protein it is protective in mice via both parenteral and intranasal inoculation and is most immunogenic when three tandem copies are fused to the N-terminus of the core protein (88-90). This is consistent with other carrier-hapten data showing that increased epitope density increases immunogenicity (91).

For intranasal delivery of an M2 vaccine an adjuvant is required to achieve good protection and good results have been achieved with LTR192G (88, 90) and CTA1-DD (89). The peptide can also be chemically conjugated to a carrier such as KLH, or the outer membrane protein complex of N. meningitides, or human papilloma virus VLPs and is protective as a vaccine in mice and other animals (92, 93).

Insofar as the M2 protein is highly conserved it is not completely without sequence divergence. The M2 ectodomain epitopes of common strains A/PR/8/34 (H1N1) and A/Aichi/68 (H3N2) were shown to be immunologically cross reactive with all other modern sequenced human strains except for A/Hong Kong/156/97 (H5N1)(92). Examination of influenza database sequences also shows similar divergence in the M2 sequence of other more recent pathogenic H5N1 human isolates such as A/Vietnam/1203/04. This finding demonstrates that a successful H5-specific pandemic vaccine incorporating M2 epitopes will need to reflect the M2 sequences that are unique to the pathogenic avian strains rather than M2 sequences currently circulating in human H1 and H3 isolates.

Additional proteins from influenza virus (other than HA, NA and M2) may be included in the VLP vaccine either by co-expression or via linkage of all or part of the additional antigen to the gag or HA polypeptides. These additional antigens include PB2, PB1, PA, nucleoprotein, matrix (M1), NS1, and NS2. These latter antigens are not generally targets of neutralizing antibody responses but may contain important epitopes recognized by T cells. T cell responses induced by a VLP vaccine to such epitopes may prove beneficial in boosting protective immunity.

Other Pathogenic Antigens

Suitable bacterial antigens include antigens associated with (e.g., synthesized by and endogenous to) any of a variety of pathogenic bacteria, including, e.g., pathogenic gram positive bacteria such as pathogenic Pasteurella species, Staphylococci species, and Streptococcus species; and gram-negative pathogens such as those of the genera Neisseria, Escherichia, Bordetella, Campylobacter, Legionella, Pseudomonas, Shigella, Vibrio, Yersinia, Salmonella, Haemophilus, Brucella, Francisella and Bacterioides. See, e.g., Schaechter, M, H. Medoff, D. Schlesinger, Mechanisms of Microbial Disease. Williams and Wilkins, Baltimore (1989)).

Suitable antigens associated with (e.g., synthesized by and endogenous to) infectious pathogenic fungi include antigens associated with infectious fungi including but not limited to: Cryptococcus neoformans, Histoplasma capsulatum, Coccidioides immitis, Blastomyces dermatitidis, and Candida albicans, Candida glabrata, Aspergillus fumigata, Aspergillus flavus, and Sporothrix schenckii.

Suitable antigens associated with (e.g., synthesized by and endogenous to) pathogenic protozoa, helminths, and other eukaryotic microbial pathogens include antigens associated with protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

Suitable antigens include antigens associated with (e.g., synthesized by and endogenous to) pathogenic microorganisms such as: Helicobacter pyloris, Borelia burgdorferi, Legionella pneumophila, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Chlamydia trachomatis, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israeli. Non-limiting examples of pathogenic E. coli strains are: ATCC No. 31618, 23505, 43886, 43892, 35401, 43896, 33985, 31619 and 31617.

Any of a variety of polypeptides or other antigens associated with intracellular pathogens may be included in the VLPs. Polypeptides and peptide epitopes associated with intracellular pathogens are any polypeptide associated with (e.g., encoded by) an intracellular pathogen, fragments of which are displayed together with MHC Class I molecule on the surface of the infected cell such that they are recognized by, e.g., bound by a T-cell antigen receptor on the surface of, a CD8.sup.+lymphocyte. Polypeptides and peptide epitopes associated with intracellular pathogens are known in the art and include, but are not limited to, antigens associated with human immunodeficiency virus, e.g., HIV gp120, or an antigenic fragment thereof; cytomegalovirus antigens; Mycobacterium antigens (e.g., Mycobacterium avium, Mycobacterium tuberculosis, and the like); Pneumocystic carinii (PCP) antigens; malarial antigens, including, but not limited to, antigens associated with Plasmodium falciparum or any other malarial species, such as 41-3, AMA-1, CSP, PFEMP-1, GBP-130, MSP-1, PFS-16, SERP, etc.; fungal antigens; yeast antigens (e.g., an antigen of a Candida spp.); toxoplasma antigens, including, but not limited to, antigens associated with Toxoplasma gondii, Toxoplasma encephalitis, or any other Toxoplasma species; Epstein-Barr virus (EBV) antigens; Plasmodium antigens (e.g., gp190/MSP1, and the like); etc.

A preferred VLP vaccine may be directed against Bacillus anthracis. Bacillus anthracis are aerobic or facultative anaerobic Gram-positive, nonmotile rods measuring 1.0 μm wide by 3.0-5.0 μm long. Under adverse conditions, B. anthracis form highly resistant endospores, which can be found in soil at sites where infected animals previously died. A preferred antigen for use in a VLP vaccine as disclosed herein is the protective antigen (PA), an 83 kDa protein that binds to receptors on mammalian cells and is critical to the ability of B. anthracis to cause disease. A more preferred antigen is the C-terminal 140 amino acid fragment of Bacillus anthracis PA which may be used to induce protective immunity in a subject against Bacillus anthracis. Other exemplary antigens for use in a VLP vaccine against anthrax are antigens from the anthrax spore (e.g., BclA), antigens from the vegetative stage of the bacterium (e.g., a cell wall antigen, capsule antigen (e.g., poly-gamma-D-glutamic acid or PGA), secreted antigen (e.g., exotoxin such as protective antigen, lethal factor, or edema factor). Another preferred antigen for use in a VLP vaccine is the tetra-saccharide containing anthrose, which is unique to B. anthracis (Daubenspeck J. M., et al. J. Biol. Chem. (2004), 279:30945). The tetra-saccharide may be coupled to a lipid raft-associated polypeptide allowing association of the antigen with the VLP vaccine.

Tumor-Associated Antigens

Any of a variety of known tumor-specific antigens or tumor-associated antigens (TAA) can be included in the VLPs. The entire TAA may be, but need not be, used. Instead, a portion of a TAA, e.g., an epitope, may be used. Tumor-associated antigens (or epitope-containing fragments thereof) which may be used in VLPs include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV18, TUAN, alpha-feto protein (AFP), OFP, CA-125, CA-50, CA-19-9, renal tumor-associated antigen G250, EGP-40 (also known as EpCAM), S100 (malignant melanoma-associated antigen), p53, and p21ras. A synthetic analog of any TAA (or epitope thereof), including any of the foregoing, may be used. Furthermore, combinations of one or more TAAs (or epitopes thereof) may be included in the composition.

Allergens

In one aspect, the antigen that is part of the VLP vaccine may be any of a variety of allergens. Allergen based vaccines may be used to induce tolerance in a subject to the allergen. Examples of an allergen vaccine involving co-precipitation with tyrosine may be found in U.S. Pat. Nos. 3,792,159, 4,070,455, and 6,440,426, each of which is hereby incorporated by reference in their entirety with particular reference to formulation of allergen vaccines.

Any of a variety of allergens can be included in VLPs. Allergens include but are not limited to environmental aeroallergens; plant pollens such as ragweed/hayfever; weed pollen allergens; grass pollen allergens; Johnson grass; tree pollen allergens; ryegrass; arachnid allergens, such as house dust mite allergens (e.g., Der p I, Der f I, etc.); storage mite allergens; Japanese cedar pollen/hay fever; mold spore allergens; animal allergens (e.g., dog, guinea pig, hamster, gerbil, rat, mouse, etc., allergens); food allergens (e.g., allergens of crustaceans; nuts, such as peanuts; citrus fruits); insect allergens; venoms: (Hymenoptera, yellow jacket, honey bee, wasp, hornet, fire ant); other environmental insect allergens from cockroaches, fleas, mosquitoes, etc.; bacterial allergens such as streptococcal antigens; parasite allergens such as Ascaris antigen; viral antigens; fungal spores; drug allergens; antibiotics; penicillins and related compounds; other antibiotics; whole proteins such as hormones (insulin), enzymes (streptokinase); all drugs and their metabolites capable of acting as incomplete antigens or haptens; industrial chemicals and metabolites capable of acting as haptens and functioning as allergens (e.g., the acid anhydrides (such as trimellitic anhydride) and the isocyanates (such as toluene diisocyanate)); occupational allergens such as flour (e.g., allergens causing Baker's asthma), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates.

Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoas); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherun elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Preferred Methods of Making VLPs

VLPs may be readily assembled by any methods available to one of skill in the art that preferably results in assembled VLPs including a gag polypeptide and a lipid-raft associated polypeptide linked to an antigen which does not naturally associate with a lipid raft. In preferred embodiments, the polypeptides may be co-expressed in any available protein expression system, preferably a cell-based system that includes raft-lipid domains in the lipids such as mammalian cell expression systems and insect cell expression systems.

Numerous examples of expression of VLPs formed using a gag polypeptide have been published demonstrating the range of expression systems available for generating VLPs. Studies with several retroviruses have demonstrated that the Gag polypeptide expressed in the absence of other viral components is sufficient for VLP formation and budding at the cell surface (Wills and Craven AIDS 5, 639-654, 1991; Zhou et al., 3. Virol. 68, 2556-2569, 1994; Morikawa et al., Virology 183, 288-297, 1991; Royer et al., Virology 184, 417-422, 1991; Gheysen et al., Cell 59, 103-112, 1989; Hughes et al., Virology 193, 242-255, 1993; Yamshchikov et al., Virology 214, 50-58, 1995). Formation of VLP upon expression of the Gag precursor in insect cells using a Baculovirus vector has been demonstrated by several groups (Delchambre et al., EMBO J. 8, 2653-2660, 1989; Luo et al., Virology 179, 874-880, 1990; Royer et al., Virology 184, 417-422, 1991; Morikawa et al., Virology 183, 288-297, 1991; Zhou et al., J. Virol. 68, 2556-2569, 1994; Gheysen et al., Cell 59, 103-112, 1989; Hughes et al., Virology 193, 242-255, 1993; Yamshchikov et al., Virology 214, 50-58, 1995). These VLPs resemble immature lentivirus particles and are efficiently assembled and released by budding from the insect cell plasma membrane.

It has been reported that the amino terminal region of the Gag precursor is a targeting signal for transport to the cell surface and membrane binding which is required for virus assembly (Yu et al., J. Virol. 66, 4966-4971, 1992; an, X et al., J. Virol. 67, 6387-6394, 1993; Zhou et al., J. Virol. 68, 2556-2569, 1994; Lee and Linial J. Virol. 68, 6644-6654, 1994; Dorfman et al., J. Virol. 68, 1689-1696, 1994; Facke et al., J. Virol. 67, 4972-4980, 1993). Assembly of recombinant HIV based VLPs that contain Gag structural proteins as well as Env glycoproteins gp120 and gp41 has been reported using a vaccinia virus expression system (Haffar et al., J. Virol. 66, 4279-4287, 1992).

Recombinant expression of the polypeptides for the VLPs requires construction of an expression vector containing a polynucleotide that encodes one or more of the polypeptides. Once a polynucleotide encoding one or more of the polypeptides has been obtained, the vector for the production of the polypeptide may be produced by recombinant DNA technology using techniques well known in the art. Thus, methods for preparing a protein by expressing a polynucleotide containing any of the VLP polypeptide-encoding nucleotide sequences are described herein. Methods which are well known to those skilled in the art can be used to construct expression vectors containing the VLP polypeptide coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a gag polypeptide and a lipid-raft associated polypeptide linked to antigen, all operably linked to one or more promoters.

The expression vector may be transferred to a host cell by conventional techniques and the transfected cells are then cultured by conventional techniques to produce the VLP polypeptide(s). Thus, the invention includes host cells containing a polynucleotide encoding one or more of the VLP polypeptides operably linked to a heterologous promoter. In preferred embodiments for the generation of VLPs, vectors encoding both the gag polypeptide and a lipid-raft associated polypeptide linked to an antigen may be co-expressed in the host cell for generation of the VLP, as detailed below.

A variety of host-expression vector systems may be utilized to express the VLP polypeptides. Such host-expression systems represent vehicles by which the VLP polypeptides may be produced to generate VLPs preferably by co-expression. A wide range of hosts may be used in construct of appropriate expression vectors and preferred host-expression systems are those hosts that have lipid rafts suitable for assembly of the VLP. These include but are not limited to microorganisms such as bacteria (e.g., E. coli, B. subtilis) transformed with recombinant bacteriophage DNA, plasmid DNA or cosmid DNA expression vectors containing VLP polypeptide coding sequences; yeast (e.g., Saccharomyces, Pichia) transformed with recombinant yeast expression vectors containing VLP polypeptide coding sequences; insect cell systems infected with recombinant virus expression vectors (e.g., baculovirus) containing VLP polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing VLP polypeptide coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, mammalian cells and more preferably insect cells are used for the expression of the VLP polypeptides, as both have raft lipid suitable for assembly of the VLPs. For example, mammalian cells such as Chinese hamster ovary cells (CHO), in conjunction with a vector such as the major intermediate early gene promoter element from human cytomegalovirus is an effective expression system for VLP polypeptides (Foecking et al., Gene 45:101 (1986); Cockett et al., Bio/Technology 8:2 (1990)).

In an insect system, Autographa californica nuclear polyhedrosis virus (AcNPV) may be used as a vector to express foreign genes. The virus grows in Spodoptera frugiperda cells. The VLP polypeptide coding sequence(s) may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the VLP polypeptide sequence(s) of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the VLP polypeptide(s) in infected hosts. (e.g., see Logan & Shenk, Proc. Natl. Acad. Sci. USA 81:355-359 (1984)). Specific initiation signals may also be required for efficient translation of inserted VLP polypeptide coding sequence(s). These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage or transport to the membrane) of protein products may be important for the generation of the VLP or function of a VLP polypeptide or additional polypeptide such as an adjuvant or additional antigen. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

The host cell may be co-transfected with two expression vectors of the invention, the first vector encoding a gag polypeptide and the second vector encoding a lipid-raft associated polypeptide linked to an antigen. The two vectors may contain identical selectable markers which enable equal expression of each VLP polypeptide. Alternatively, a single vector may be used which encodes, and is capable of expressing, both the gag polypeptide and the lipid-raft associated polypeptide linked to an antigen

Once a VLP has been produced by a host cell, it may be purified by any method known in the art for purification of a polypeptide, for example, by chromatography (e.g., ion exchange, affinity, particularly by affinity for any affinity purification tags added to the polypeptide, and sizing column chromatography), centrifugation, differential solubility, or by any other standard technique for the purification of proteins or other macromolecules. In addition, the VLP polypeptide can be fused to heterologous polypeptide sequences described herein or otherwise known in the art, to facilitate purification of the VLP. After purification, additional elements such as additional antigens or adjuvants may be physically linked to the VLP either through covalent linkage to the VLP polypeptides or by other non-covalent linkages mechanism. In preferred embodiments where the VLP polypeptides are co-expressed in a host cell that has raft-lipid domains such as mammalian cells and insect cells, the VLPs will self assemble and release allowing purification of the VLPs by any of the above methods.

Preferred Methods of Using VLPs

Formulations

A preferred use of the VLPs described herein is as a vaccine preparation. Typically, such vaccines are prepared as injectables either as liquid solutions or suspensions; solid forms suitable for solution in, or suspension in, liquid prior to injection may also be prepared. Such preparations may also be emulsified or produced as a dry powder. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain auxiliary substances such as wetting or emulsifying agents, pH buffering agents, or adjuvants which enhance the effectiveness of the vaccines.

Vaccines may be conventionally administered parenterally, by injection, for example, either subcutaneously, transcutaneously, intradermally, subdermally or intramuscularly. Additional formulations which are suitable for other modes of administration include suppositories and, in some cases, oral, intranasal, buccal, sublinqual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. In certain embodiments, a low melting wax, such as a mixture of fatty acid glycerides or cocoa butter is first melted and the VLPs described herein are dispersed homogeneously, for example, by stirring. The molten homogeneous mixture is then poured into conveniently sized molds, allowed to cool, and to solidify.

Formulations suitable for intranasal delivery include liquids and dry powders. Formulations include such normally employed excipients as, for example, pharmaceutical grades of mannitol, lactose, sucrose, trehalose, and chitosan. Mucosadhesive agents such as chitosan can be used in either liquid or powder formulations to delay mucocilliary clearance of intranasally-administered formulations. Sugars such as mannitol and sucrose can be used as stability agents in liquid formulations and as stability and bulking agents in dry powder formulations. In addition, adjuvants such as monophosphoryl lipid A (MPL) and, by way of example but not limitation, double stranded poly (I:C), poly inosinic acid, CpG-containing oligonucleotides, imiquimod, cholera toxin and its derivative, heat labile enterotoxin and its derivative and many of the adjuvants listed throughout the specification, can be used in both liquid and dry powder formulations as an immunostimulatory adjuvant.

Formulations suitable for oral delivery include liquids, solids, semi-solids, gels, tablets, capsules, lozenges, and the like. Formulations suitable for oral delivery include tablets, lozenges, capsules, gels, liquids, food products, beverages, nutraceuticals, and the like. Formulations include such normally employed excipients as, for example, pharmaceutical grades of mannitol, lactose, starch, magnesium stearate, sodium saccharine, cellulose, magnesium carbonate, and the like. Other VLP vaccine compositions may take the form of solutions, suspensions, pills, sustained release formulations or powders and contain 10-95% of active ingredient, preferably 25-70%. For oral formulations, cholera toxin is an interesting formulation partner (and also a possible conjugation partner).

The VLP vaccines when formulated for vaginal administration may be in the form of pessaries, tampons, creams, gels, pastes, foams or sprays. Any of the foregoing formulations may contain agents in addition to VLPs, such as carriers, known in the art to be appropriate.

In some embodiments, the VLP vaccine may be formulated for systemic or localized delivery. Such formulations are well known in the art. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Systemic and localized routes of administration include, e.g., transcutaneous, intradermal, topical application, intravenous, intramuscular, etc.

The VLPs may be formulated into the vaccine including neutral or salt-based formulations. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines may be administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2000 μg (even though higher amounts in the 1-10 mg range are contemplated), such as in the range from about 0.5 μg to 1000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the vaccine formulations will be sufficiently immunogenic as a vaccine by themselves, but for some of the others the immune response will be enhanced if the vaccine further includes an adjuvant substance.

Delivery agents that improve mucoadhesion can also be used to improve delivery and immunogenicity especially for intranasal, oral or lung based delivery formulations. One such compound, chitosan, the N-deacetylated form of chitin, is used in many pharmaceutical formulations (32). It is an attractive mucoadhesive agent for intranasal vaccine delivery due to its ability to delay mucociliary clearance and allow more time for mucosal antigen uptake and processing (33, 34). In addition, it can transiently open tight junctions which may enhance transepithelial transport of antigen to the NALT. In a recent human trial, a trivalent inactivated influenza vaccine administered intranasally with chitosan but without any additional adjuvant yielded seroconversion and HI titers that were only marginally lower than those obtained following intramuscular inoculation (33).

Chitosan can also be formulated with adjuvants that function well intranasally such as the genetically detoxified E. coli heat-labile enterotoxin mutant LTK63. This adds an immunostimulatory effect on top of the delivery and adhesion benefits imparted by chitosan resulting in enhanced mucosal and systemic responses (35).

Finally, it should be noted that chitosan formulations can also be prepared in a dry powder format that has been shown to improve vaccine stability and result in a further delay in mucociliary clearance over liquid formulations (42). This was seen in a recent human clinical trial involving an intranasal dry powder diphtheria toxoid vaccine formulated with chitosan in which the intranasal route was as effective as the traditional intramuscular route with the added benefit of secretory IgA responses (43). The vaccine was also very well tolerated. Intranasal dry powdered vaccines for anthrax containing chitosan and MPL induce stronger responses in rabbits than intramuscular inoculation and are also protective against aerosol spore challenge (44).

Intranasal vaccines represent a preferred formulation as they can affect the upper and lower respiratory tracts in contrast to parenterally administered vaccines which are better at affecting the lower respiratory tract. This can be beneficial for inducing tolerance to allergen-based vaccines and inducing immunity for pathogen-based vaccines.

In addition to providing protection in both the upper and lower respiratory tracts, intranasal vaccines avoid the complications of needle inoculations and provide a means of inducing both mucosal and systemic humoral and cellular responses via interaction of particulate and/or soluble antigens with nasopharyngeal-associated lymphoid tissues (NALT) (16-19). The intranasal route has been historically less effective than parenteral inoculation, but the use of VLPs, novel delivery formulations, and adjuvants are beginning to change the paradigm. Indeed, VLPs containing functional hemagglutinin polypeptides may be especially well suited for intranasal delivery due to the abundance of sialic acid-containing receptors in the nasal mucosa resulting in the potential for enhanced HA antigen binding and reduced mucociliary clearance.

Adjuvants

Various methods of achieving adjuvant effect for vaccines are known and may be used in conjunction with the VLPs disclosed herein. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

In some embodiments, a VLP vaccine includes the VLP in admixture with at least one adjuvant, at a weight-based ratio of from about 10:1 to about 10¹⁰:1 VLP:ajuvant, e.g., from about 10:1 to about 100:1, from about 100:1 to about 10³:1, from about 10³:1 to about 10⁴:1, from about 10⁴:1 to about 10⁵:1, from about 10⁵:1 to about 10⁶:1, from about 10⁶:1 to about 10⁷:1, from about 10⁷:1 to about 10⁸:1, from about 10⁸:1 to about 10⁹:1, or from about 10⁹:1 to about 10¹⁰:1 VLP:adjuvant. One of skill in the art can readily determine the appropriate ratio through information regarding the adjuvant and routine experimentation to determine optimal ratios. Admixtures of VLPs and adjuvants as disclosed herein may include any form of combination available to one of skill in the art including, without limitation, mixture of separate VLPs and adjuvants in the same solution, covalently linked VLPs and adjuvants, ionically linked VLPs and adjuvants, hydrophobically linked VLPs and adjuvants (including being embedded partially or fully in the VLP membrane), hydrophilically linked VLPs and adjuvants, and any combination of the foregoing.

Preferred examples of adjuvants are polypeptide adjuvants that may be readily added to the VLPs described herein by co-expression with the VLP polypeptides or fusion with the VLP polypeptides to produce chimeric polypeptides. Bacterial flagellin, the major protein constituent of flagella, is a preferred adjuvant which has received increasing attention as an adjuvant protein because of its recognition by the innate immune system by the toll-like receptor TLR5 (65). Flagellin signaling through TLR5 has effects on both innate and adaptive immune functions by inducing DC maturation and migration as well as activation of macrophages, neutrophils, and intestinal epithelial cells resulting in production of proinflammatory mediators (66-72).

TLR5 recognizes a conserved structure within flagellin monomers that is unique to this protein and is required for flagellar function, precluding its mutation in response to immunological pressure (73). The receptor is sensitive to a 100 fM concentration but does not recognize intact filaments. Flagellar disassembly into monomers is required for binding and stimulation.

As an adjuvant, flagellin has potent activity for induction of protective responses for heterologous antigens administered either parenterally or intranasally (66, 74-77) and adjuvant effects for DNA vaccines have also been reported (78). A Th2 bias is observed when flagellin is employed which would be appropriate for a respiratory virus such as influenza but no evidence for IgE induction in mice or monkeys has been observed. In addition, no local or systemic inflammatory responses have been reported following intranasal or systemic administration in monkeys (74). The Th2 character of responses elicited following use of flagellin is somewhat surprising since flagellin signals through TLR5 in a MyD88-dependent manner and all other MyD88-dependent signals through TLRs have been shown to result in a Th1 bias (67, 79). Importantly, pre-existing antibodies to flagellin have no appreciable effect on adjuvant efficacy (74) making it attractive as a multi-use adjuvant.

A common theme in many recent intranasal vaccine trials is the use of adjuvants and/or delivery systems to improve vaccine efficacy. In one such study an influenza H3 vaccine containing a genetically detoxified E. coli heat-labile enterotoxin adjuvant (LT R192G) resulted in heterosubtypic protection against H5 challenge but only following intranasal delivery. Protection was based on the induction of cross neutralizing antibodies and demonstrated important implications for the intranasal route in development of new vaccines (22).

Cytokines, colony-stimulating factors (e.g., GM-CSF, CSF, and the like); tumor necrosis factor; interleukin-2, -7, -12, interferons and other like growth factors, may also be used as adjuvants and are also preferred as they may be readily included in the VLP vaccine by admixing or fusion with the VLP polypeptides.

In some embodiments, the VLP vaccine compositions disclosed herein may include other adjuvants that act through a Toll-like receptor such as a nucleic acid TLR9 ligand comprising a CpG oligonucleotide; an imidazoquinoline TLR7 ligand; a substituted guanine TLR7/8 ligand; other TLR7 ligands such as Loxoribine, 7-deazadeoxyguanosine, 7-thia-8-oxodeoxyguanosine, double stranded poly (I:C), poly inosinic acid, Imiquimod (R-837), and Resiquimod (R-848); or a TLR4 agonist such as MPL® or synthetic derivatives.

Certain adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; MPL; and an encapsulating adjuvant.

Additional examples of adjuvants include agents such as aluminum salts such as hydroxide or phosphate (alum), commonly used as 0.05 to 0.1 percent solution in buffered saline (see, e.g., Nicklas (1992) Res. Immunol. 143:489-493), admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

DDA (dimethyldioctadecylammonium bromide) is an interesting candidate for an adjuvant, but also Freund's complete and incomplete adjuvants as well as quillaja saponins such as QuilA and QS21 are interesting. Further possibilities include poly[di(earboxylatophenoxy)phosphazene (PCPP) derivatives of lipopolysaccharides such as monophosphoryl lipid A (MPL®), muramyl dipeptide (MDP) and threonyl muramyl dipeptide (tMDP). The lipopolysaccharide based adjuvants are preferred for producing a predominantly Th1-type response including, for example, a combination of monophosphoryl lipid A, preferably 3-de-O-acylated monophosphoryl lipid A, together with an aluminum salt. MPL® adjuvants are available from GlaxoSmithKline (see, for example, U.S. Pat. Nos. 4,436,727; 4,877,611; 4,866,034 and 4,912,094, each of which is incorporated by reference in their entirety with particular reference to their lipopolysaccharides related teachings).

Liposome formulations are also known to confer adjuvant effects, and therefore liposome adjuvants are preferred examples in conjunction with the VLPs.

Immunostimulating complex matrix type (ISCOM® matrix) adjuvants are preferred choices according to the invention, especially since it has been shown that this type of adjuvants are capable of up-regulating MHC Class II expression by APCs. An ISCOM matrix consists of (optionally fractionated) saponins (triterpenoids) from Quillaja saponaria, cholesterol, and phospholipid. When admixed with the immunogenic protein such as in the VPLs, the resulting particulate formulation is what is known as an ISCOM particle where the saponin may constitute 60-70% w/w, the cholesterol and phospholipid 10-15% w/w, and the protein 10-15% w/w. Details relating to composition and use of immunostimulating complexes can for example be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

The saponins, whether or not in the form of iscoms, that may be used in the adjuvant combinations with the VLP vaccines disclosed herein include those derived from the bark of Quillaja Saponaria Molina, termed Quil A, and fractions thereof, described in U.S. Pat. No. 5,057,540 (which is incorporated by reference herein in its entirety with particular reference to the fractions of Quil A and methods of isolation and use thereof) and “Saponins as vaccine adjuvants”, Kensil, C. R., Crit. Rev Ther Drug Carrier Syst, 1996, 12 (1-2):1-55; and EP 0 362 279 B1. Particularly preferred fractions of Quil A are QS21, QS7, and QS17.

β-Escin is another preferred haemolytic saponins for use in the adjuvant compositions of the present invention. Escin is described in the Merck index (12th ed: entry 3737) as a mixture of saponins occurring in the seed of the horse chestnut tree, Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin have been purified and shown to be biologically active (Yoshikawa M, et al. (Chem Pharm Bull (Tokyo) 1996 August; 44(8):1454-1464)). β-escin is also known as aescin.

Another preferred haemolytic saponin for use in the present invention is Digitonin. Digitonin is described in the Merck index (12.sup.th Edition, entry 3204) as a saponin, being derived from the seeds of Digitalis purpurea and purified according to the procedure described Gisvold et al., J. Am. Pharm. Assoc., 1934, 23, 664; and Ruhenstroth-Bauer, Physiol. Chem., 1955, 301, 621. Its use is described as being a clinical reagent for cholesterol determination.

Another interesting (and thus, preferred) possibility of achieving adjuvant effect is to employ the technique described in Gosselin et al., 1992 (which is hereby incorporated by reference herein). In brief, the presentation of a relevant antigen such as an antigen of the present invention can be enhanced by conjugating the antigen to antibodies (or antigen binding antibody fragments) against the F_{C} receptors on monocytes/macrophages. Especially conjugates between antigen and anti-F_{C}RI have been demonstrated to enhance immunogenicity for the purposes of vaccination. The antibody may be conjugated to the VLP after generation or as a part of the generation including by expressing as a fusion to any one of the VLP polypeptides.

Other possibilities involve the use of the targeting and immune modulating substances (i.e. cytokines). In addition, synthetic inducers of cytokines such as poly I:C may also be used.

Suitable mycobacterial derivatives may be selected from the group consisting of muramyl dipeptide, complete Freund's adjuvant, RIBI, (Ribi ImmunoChem Research Inc., Hamilton, Mont.) and a diester of trehalose such as TDM and TDE.

Examples of suitable immune targeting adjuvants include CD40 ligand and CD40 antibodies or specifically binding fragments thereof (cf. the discussion above), mannose, a Fab fragment, and CTLA-4.

Examples of suitable polymer adjuvants include a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12-15, 1998, Seascape Resort, Aptos, Calif.”

Oligonucleotides may be used as adjuvants in conjunction with the VLP vaccines and preferably contain two or more dinucleotide CpG motifs separated by at least three or more preferably at least six or more nucleotides. CpG-containing oligonucleotides (in which the CpG dinucleotide is unmethylated) induce a predominantly Th1 response. Such oligonucleotides are well known and are described, for example, in WO 96/02555, WO 99/33488 and U.S. Pat. Nos. 6,008,200 and 5,856,462, each of which is hereby incorporated by reference in their entirety with particular reference to methods of making and using CpG oligonucleotides as adjuvants.

Such oligonucleotide adjuvants may be deoxynucleotides. In a preferred embodiment the nucleotide backbone in the oligonucleotide is phosphorodithioate, or more preferably a phosphorothioate bond, although phosphodiester and other nucleotide backbones such as PNA are within the scope of the invention including oligonucleotides with mixed backbone linkages. Methods for producing phosphorothioate oligonucleotides or phosphorodithioate are described in U.S. Pat. No. 5,666,153, U.S. Pat. No. 5,278,302 and WO95/26204, each of which are hereby incorporated by reference in their entirety with particular reference to the phosphorothioate and phosphorodithioate teachings.

Examples of preferred oligonucleotides have the following sequences. The sequences preferably contain phosphorothioate modified nucleotide backbones.

(SEQ ID NO: 1)

OLIGO 1: TCC ATG ACG TTC CTG ACG TT (CpG 1826)

(SEQ ID NO: 2)

OLIGO 2: TCT CCC AGC GTG CGC CAT (CpG 1758)

(SEQ ID NO: 3)

OLIGO 3: ACC GAT GAC GTC GCC GGT GAC GGC ACC ACG

(SEQ ID NO: 4)

OLIGO 4: TCG TCG TTT TGT CGT TTT GTC GTT (CpG
2006)

(SEQ ID NO: 5)

OLIGO 5: TCC ATG ACG TTC CTG ATG CT (CpG 1668)

Alternative preferred CpG oligonucleotides include the above sequences with inconsequential deletions or additions thereto. The CpG oligonucleotides as adjuvants may be synthesized by any method known in the art (e.g., EP 468520). Preferably, such oligonucleotides may be synthesized utilizing an automated synthesizer. Such oligonucleotide adjuvants may be between 10-50 bases in length. Another adjuvant system involves the combination of a CpG-containing oligonucleotide and a saponin derivative particularly the combination of CpG and QS21 is disclosed in WO 00/09159.

Many single or multiphase emulsion systems have been described. One of skill in the art may readily adapt such emulsion systems for use with VLPs so that the emulsion does not disrupt the VLP's structure. Oil in water emulsion adjuvants per se have been suggested to be useful as adjuvant compositions (EPO 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B).

The oil emulsion adjuvants for use with the VLP vaccines described herein may be natural or synthetic, and may be mineral or organic. Examples of mineral and organic oils will be readily apparent to the man skilled in the art.

In order for any oil in water composition to be suitable for human administration, the oil phase of the emulsion system preferably includes a metabolisable oil. The meaning of the term metabolisable oil is well known in the art. Metabolisable can be defined as “being capable of being transformed by metabolism” (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils are also part of this invention and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil virtue of the fact that it is an intermediate in the biosynthesis of cholesterol (Merck index, 10th Edition, entry no. 8619).

Particularly preferred oil emulsions are oil in water emulsions, and in particular squalene in water emulsions.

In addition, the most preferred oil emulsion adjuvants of the present invention include an antioxidant, which is preferably the oil α-tocopherol (vitamin E, EP 0 382 271 B1).

WO 95/17210 and WO 99/11241 disclose emulsion adjuvants based on squalene, α-tocopherol, and TWEEN 80, optionally formulated with the immunostimulants QS21 and/or 3D-MPL. WO 99/12565 discloses an improvement to these squalene emulsions with the addition of a sterol into the oil phase. Additionally, a triglyceride, such as tricaprylin (C27H50O6), may be added to the oil phase in order to stabilise the emulsion (WO 98/56414).

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges. The amounts of the components present in the oil emulsions of the present invention are conventionally in the range of from 2 to 10% oil, such as squalene; and when present, from 2 to 10% alpha tocopherol; and from 0.3 to 3% surfactant, such as polyoxyethylene sorbitan monooleate. Preferably the ratio of oil: alpha tocopherol is equal or less than 1 as this provides a more stable emulsion. Span 85 may also be present at a level of about 1%. In some cases it may be advantageous that the VLP vaccines disclosed herein will further contain a stabiliser.

The method of producing oil in water emulsions is well known to the man skilled in the art. Commonly, the method includes the step of mixing the oil phase with a surfactant such as a PBS/TWEEN80® solution, followed by homogenisation using a homogenizer, it would be clear to a man skilled in the art that a method comprising passing the mixture twice through a syringe needle would be suitable for homogenising small volumes of liquid. Equally, the emulsification process in microfluidiser (M110S microfluidics machine, maximum of 50 passes, for a period of 2 minutes at maximum pressure input of 6 bar (output pressure of about 850 bar)) could be adapted by the man skilled in the art to produce smaller or larger volumes of emulsion. This adaptation could be achieved by routine experimentation comprising the measurement of the resultant emulsion until a preparation was achieved with oil droplets of the required diameter.

The VLP vaccine preparations disclosed herein may be used to protect or treat a mammal or bird susceptible to, or suffering from a viral infection, by means of administering the vaccine by intranasal, intramuscular, intraperitoneal, intradermal, transdermal, intravenous, or subcutaneous administration. Methods of systemic administration of the vaccine preparations may include conventional syringes and needles, or devices designed for ballistic delivery of solid vaccines (WO 99/27961), or needleless pressure liquid jet device (U.S. Pat. No. 4,596,556; U.S. Pat. No. 5,993,412), or transdermal patches (WO 97/48440; WO 98/28037). The VLP vaccines may also be applied to the skin (transdermal or transcutaneous delivery WO 98/20734; WO 98/28037). The VLP vaccines disclosed herein therefore includes a delivery device for systemic administration, pre-filled with the VLP vaccine or adjuvant compositions. Accordingly there is provided a method for inducing an immune response in an individual preferably mammal or bird, comprising the administration of a vaccine comprising any of the VLP compositions described herein and optionally including an adjuvant and/or a carrier, to the individual, wherein the vaccine is administered via the parenteral or systemic route.

Preferrably the vaccine preparations of the present invention may be used to protect or treat a mammal or bird susceptible to, or suffering from a viral infection, by means of administering the vaccine via a mucosal route, such as the oral/alimentary or nasal route. Alternative mucosal routes are intravaginal and intra-rectal. The preferred mucosal route of administration is via the nasal route, termed intranasal vaccination. Methods of intranasal vaccination are well known in the art, including the administration of a droplet, spray, or dry powdered form of the vaccine into the nasopharynx of the individual to be immunised. Nebulised or aerosolised vaccine formulations are therefore preferred forms of the VLP vaccines disclosed herein. Enteric formulations such as gastro resistant capsules and granules for oral administration, suppositories for rectal or vaginal administration are also formulations of the VLP vaccines disclosed herein.

The preferred VLP vaccine compositions disclosed herein, represent a class of mucosal vaccines suitable for application in humans to replace systemic vaccination by mucosal vaccination.

The VLP vaccines may also be administered via the oral route. In such cases the pharmaceutically acceptable excipient may also include alkaline buffers, or enteric capsules or microgranules. The VLP vaccines may also be administered by the vaginal route. In such cases, the pharmaceutically acceptable excipients may also include emulsifiers, polymers such as CARBOPOL®, and other known stabilisers of vaginal creams and suppositories. The VLP vaccines may also be administered by the rectal route. In such cases the excipients may also include waxes and polymers known in the art for forming rectal suppositories.

Alternatively the VLP vaccines formulations may be combined with vaccine vehicles composed of chitosan (as described above) or other polycationic polymers, polylactide and polylactide-coglycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM.

Additional illustrative adjuvants for use in the pharmaceutical and vaccine compositions using VLPs as described herein include SAF (Chiron, Calif., United States), MF-59 (Chiron, see, e.g., Granoff et al. (1997) Infect Immun. 65 (5):1710-1715), the SBAS series of adjuvants (e.g., SB-AS2 (SmithKline Beecham adjuvant system #2; an oil-in-water emulsion containing MPL and QS21); SBAS-4 (SmithKline Beecham adjuvant system #4; contains alum and MPL), available from SmithKline Beecham, Rixensart, Belgium), Detox (Enhanzyn®) (GlaxoSmithKline), RC-512, RC-522, RC-527, RC-529, RC-544, and RC-560 (GlaxoSmithKline) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

Other examples of adjuvants include, but are not limited to, Hunter's TiterMax® adjuvants (CytRx Corp., Norcross, Ga.); Gerbu adjuvants (Gerbu Biotechnik GmbH, Gaiberg, Germany); nitrocellulose (Nilsson and Larsson (1992) Res. Immunol. 143:553-557); alum (e.g., aluminum hydroxide, aluminum phosphate) emulsion based formulations including mineral oil, non-mineral oil, water-in-oil or oil-in-water emulsions, such as the Seppic ISA series of Montamide adjuvants (e.g., ISA-51, ISA-57, ISA-720, ISA-151, etc.; Seppic, Paris, France); and PROVAX® (IDEC Pharmaceuticals); OM-174 (a glucosamine disaccharide related to lipid A); Leishmania elongation factor; non-ionic block copolymers that form micelles such as CRL 1005; and Syntex Adjuvant Formulation. See, e.g., O'Hagan et al. (2001) Biomol Eng. 18(3):69-85; and “Vaccine Adjuvants: Preparation Methods and Research Protocols” D. O'Hagan, ed. (2000) Humana Press.

Other preferred adjuvants include adjuvant molecules of the general formula

HO(CH₂CH₂O)ₙ-A-R, (I)

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C.sub.1-50, preferably C.sub.4-C.sub.20 alkyl and most preferably C.sub.12 alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12.sup.th edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described above.

Further examples of suitable pharmaceutically acceptable excipients for use with the VLP vaccines disclosed herein include water, phosphate buffered saline, isotonic buffer solutions.

This invention will be better understood by reference to the following non-limiting Examples. As described herein, the invention includes chimeric VLPs incorporating any type of lipid raft-associated polypeptide linked to an antigen which does not naturally associate with a lipid raft. The following Examples describe a representative embodiment of the invention, chimeric VLPs with influenza antigens.

### EXAMPLE 1

### Production of a Chimeric Influenza VLP

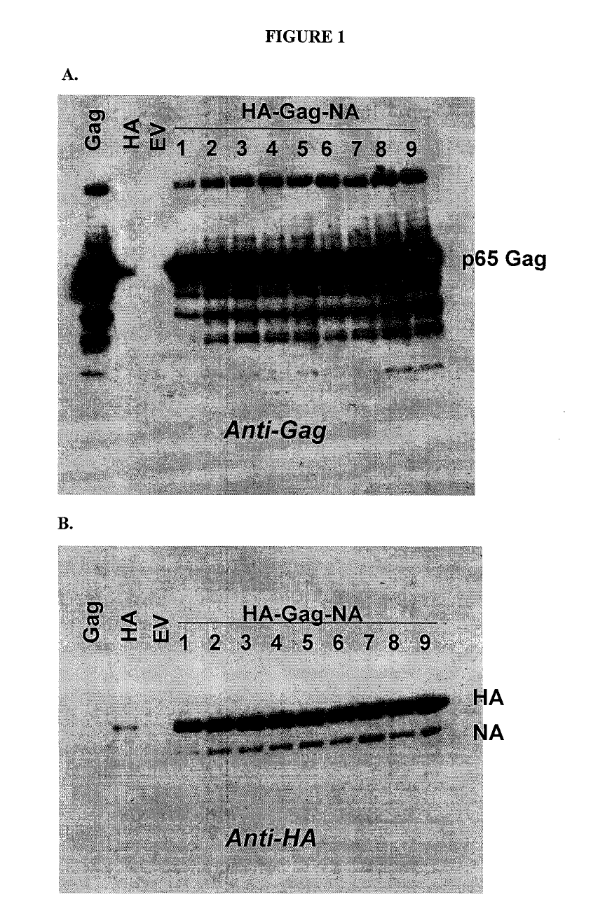

The MLV gag coding sequence was obtained by PCR from plasmid pAMS (ATCC) containing the entire Moloney murine leukemia virus amphotropic proviral sequence. The gag coding sequence was inserted into pFastBac1 (Invitrogen) behind the polyhedron promoter and the resulting plasmid was transformed into DH10Bac competent cells for recombination into the baculovirus genome. High molecular weight bacmid DNA was then purified and transfected into Sf9 cells for generation of a gag-expressing recombinant baculovirus. Two other recombinant baculoviruses encoding the hemagglutinin and neuraminidase, respectively, of A/PR/8/34 (H1N1) were produced in a similar fashion after RT-PCR cloning of the HA and NA coding sequences from virus RNA. Finally, a single baculovirus vector encoding all three products (HA-gag-NA) was produced by combining the HA, gag, and NA expression units (polyhedron promoter—coding sequence—polyA site) from individual pFastBac1 plasmids into a single pFastBac1 vector. For initial analysis, recombinant baculoviruses encoding gag or HA or gag-HA-NA were infected into Sf9 cells in 6 well plates at an MOI of >1. Three days following infection, medium supernatants were clarified of debris then pelleted at 100,000×g through a 20% sucrose cushion. Pellets were analyzed by Western blot analysis using gag and H1N1-specific antisera (See FIGS. 1A and B).

The left three lanes on each blot in FIGS. 1A and B, respectively, show the results of infecting Sf9 cells with separate gag or HA or control (EV=empty vector) baculoviruses prior to harvesting the medium. As expected, infection with a gag-only baculovirus results in significant amounts of gag antigen in the high molecular weight medium fraction due to VLP budding (FIG. 1A, lane “Gag”). In contrast, infection with an HA only baculovirus, results in little HA released into the medium on its own (FIG. 1B, lane “HA”). However, infection of Sf9 cells with a HA-gag-NA triple vector results in significant amounts of both gag and HA appearing in the 100,000×g fraction (lanes 1-9, FIGS. 1A and B) showing that gag expression can pull HA out of the cell.

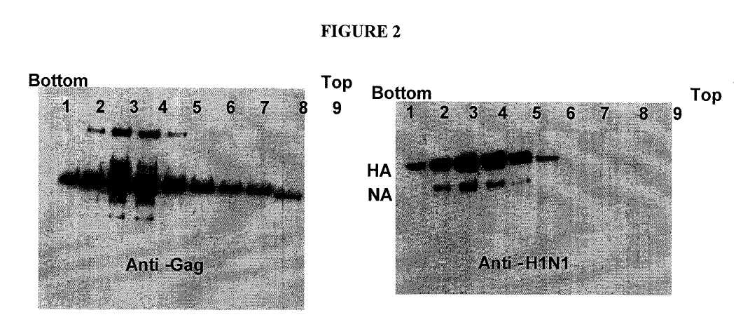

The FIGS. 2A and B show the results of recentrifugation of pelleted HA-gag-NA VLPs on a 20-60% sucrose step gradient followed by Western blot analysis of individual gradient fractions. Both gag and HA peak in the same fraction demonstrating coincident banding at a density of approximately 1.16 g/ml which indicates that the gag and HA were in VLPs.

### EXAMPLE 2

### Production, Characterization and Immunogenicity Testing of HA-gag-NA VLPs Containing an Anthrax PA Epitope Attached to HA

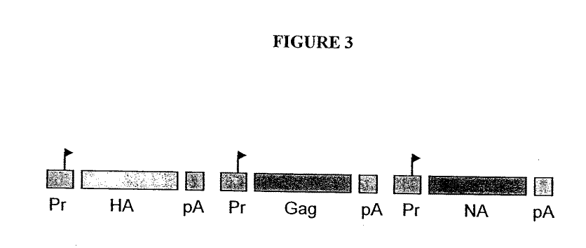

As described in Example 1, individual baculoviruses expressing MLV gag and the HA and NA products of A/PR/8/34 have been produced. In addition a triple expression recombinant baculovirus encoding all three products has also been constructed and sucrose density gradient centrifugation of pelleted medium supernatants from infected insect cells showed coincident banding of gag and HA as detected by Western blotting indicating that VLPs with HA had formed. The arrangement of coding sequences in the triple expression vector is shown in FIG. 3 in which the HA, gag, and NA coding elements are arranged in a head-to-tail fashion, each with its own promoter (Pr) and polyadenylation sequence (pA). Combining all coding sequences into a single baculovirus avoids the need to perform co-infections with separate viruses and the associated difficulties of achieving consistent multiplicities of infection of three separate viruses.

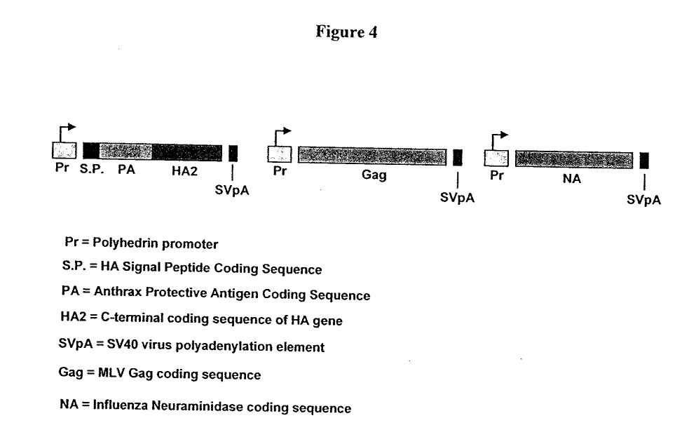

The VLP vaccine in this Example 2 will be generated in a similar fashion except that the HA gene will be modified by replacing most of its HA immunological determinants with that of the protective antigen (PA) of B. anthracis. This will be accomplished by replacing the coding sequence for the HA1 portion of the HA gene (amino acid positions 18-343) with that of a 140 amino acid C-terminal fragment of PA. The PA coding sequence will be inserted into the HA gene between the HA signal peptide coding sequence (amino acid positions 1-17) and the HA2 coding sequence (amino acid positions 344-565) as a direct replacement of the HA1 coding sequence (amino acid positions 18-343) (FIG. 4). The triple baculovirus expression vector containing the modified PA-HA gene will express the MLV gag polypeptide, the NA polypeptide, and the modified PA-HA polypeptide containing the replacement element. When the triple expression vector is used to infect Sf9 cells in culture, VLPs containing the anthrax PA epitope will be observed in the culture medium because the modified PA-HA polypeptide will still retain lipid raft homing sequences and will be incorporated into particles budding from lipid raft domains. Evidence for this will be shown by harvesting culture medium from cells infected with the (PA)HA-gag-NA triple expression vector, clearing the medium of debris, and collecting chimeric VLPs by centrifugation at 100,000×g over a 20% sucrose cushion. Evidence for PA incorporation into the VLPs will be obtained by Western blot analysis using a PA-specific antibody. By definition, material that sediments through a 20% sucrose cushion under these conditions is particulate in nature, providing evidence of VLP formation.

Size Exclusion Chromatography:

Sucrose density gradient purified VLPs will be subjected to size exclusion chromatography using Sepharose CL-4B and fractions will be monitored for MLV gag and anthrax PA epitopes by Western blot. VLPs will elute in the void volume and will contain MLV gag, NA, and the PA-modified HA.

Electron Microscopy:

VLP samples from sucrose gradients will be treated with 2% glutaraldehyde, adsorbed onto EM grids, negatively stained with sodium phosphotungstate and examined by electron microscopy.

Immunogenicity:

Chimeric VLP immunogenicity (VLPs containing the anthrax PA-modified HA) will be evaluated in female Balb/c mice using intranasal chitosan/MPL formulations similar to the anthrax protective antigen (PA) formulation described in reference (44). VLPs will be purified by pelleting VLP-containing culture medium through 20% sucrose cushions at 100,000×g for 1 hour after which they will be resuspended in Tris-buffered saline and banded on 20-60% sucrose density gradients. VLP-containing fractions will be identified by SDS PAGE or Western blot and pooled. VLPs samples will be dialyzed into PBS and concentrated using centrifuge microconcentrators or by centrifugation at 100,000×g.

For immunization, liquid formulations (15 μl) containing 40 μg chitosan, 20 μg VLP (based on gag), and 5 μg MPL will be divided between the two nostrils for a single immunization. Animals will be lightly anesthetized with isoflurane prior to intranasal dosing at 0 and 4 weeks. VLPs will also be formulated in PBS with MPL or cholera toxin for intraperitoneal inoculation as a positive control. Systemic IgG responses will be monitored by ELISA for immune responses specific for PA. At the time of sacrifice, broncoaveolar lavage samples will also be collected for determination of PA-specific IgA responses.

Typical immunization experiments in this Example 2 and the Examples below will employ a minimum of eight mice per group which will provide a reasonable probability of achieving statistical significance as shown using Student's unpaired t-test. Immunizations will typically entail primary and booster inoculations spaced four weeks apart with blood sampling occurring 10-14 days following each immunization. As stated above, broncoaveolar lavage samples will be collected from sacrificed animals for IgA determination.

### EXAMPLE 3

### Production and Immunogenicity Testing of Enhanced VLPs

This Example 3 will demonstrate the enhancements of the VLPs for improved immunogenicity and protection by incorporation of the TLR5 agonist flagellin to boost the strength of adaptive immune responses to the anthrax PA epitope attached to NA.

Adjuvant Effects Due to Flagellin Incorporation:

The flagellin coding sequence was recently cloned from S. typimurium genomic DNA and inserted at the 3′ end of the gag coding sequence just 5′ to the termination codon. The flagellin coding sequence will also be inserted at the N-terminus of the A/PR/8/34 HA coding sequence using a PstI site located at the boundary between the signal peptide and mature coding sequences. Insertions at this location in HA lead to proper expression of chimeric HA molecules with expected molecular weight increases as demonstrated by SDS PAGE. The NA polypeptide will have the 140 amino acid anthrax C-terminal PA domain fused to its C-terminus. The C-terminus of NA is found on the outside of the particle envelope such that C-terminal extensions will be expected to be exposed on the outside of the particle. Flagellin-modified gag and HA coding sequences will be used to generate triple baculovirus recombinants (HA-gag-NA(PA)). Recombinant baculoviruses encoding VLPs with flagellin-modified gag or flagellin-modified HA will be produced and used to generate VLPs for immunogenicity testing versus basic VLPs lacking flagellin sequences (All VLPs will contain the PA-modified NA as the target epitope and will either contain or lack flagellin sequences attached to gag or HA. As stated in Example 2, all immunization experiments will employ primary and booster inoculations spaced four weeks apart. Immunological readouts will be via ELISA assays as described above, examining both systemic IgG and mucosal IgA responses.

Because the HA and gag insertion sites for flagellin incorporation are outside and inside the VLP, respectively, different degrees of adjuvant effects will be observed. Flagellin insertion at the N-terminus of HA will result in easy access of flagellin to TLR5 receptors on cells in the epithelial mucosa. In contrast, the gag site of insertion will result in different access. VLP binding to cells and internalization via the normal influenza virus entry pathway will result in the deposition of the gag-flagellin product within the cell. This will result in differential TLR5-mediated adjuvant effects between the gag-flagellin and the HA-flagellin constructs. Since the ability of VLPs to bind to and enter mucosal epithelial cells may in itself have an effect on immunogenicity, we will perform VLP immunogenicity studies of flagellin-modified and normal VLPs with and without TPCK-trypsin treatment. HA cleavage of trypsin-treated VLPs will be confirmed by Western blot prior to the initiation of immunogenicity studies examining the importance of VLP entry. In addition, the ability of trypsin-treated VLPs to fuse with and enter cells will be examined by in vitro fluorescence microscopy studies employing VLPs containing a green fluorescent protein (GFP)-modified gag product. It has already been shown that MLV gag can be modified at its C-terminus with GFP without abrogation of its budding activity (60).

The use of subfragments of the flagellin coding sequence to maximize gag budding activity by eliminating much of the non-TLR5 binding regions of flagellin will also be tested. Recent mapping of the TLR5 recognition sites within the flagellin monomer will facilitate this effort (73).

TABLE 1
Example 3: Animal studies

| | |
|---|---|
| Flagellin-enhanced VLP Immunogenicity test | # of mice |

| | |
|---|---|
| Group 1: Neg. control | 8 |
| Group 2: VLP w/ gag-flagellin - (HA-gag(flag)-NA(PA)) | 8 |
| Group 3: VLP w/ HA-flagellin - (HA(flag)-gag-NA(PA)) | 8 |
| Group 4: Basic VLP - (HA-gag-NA(PA)) | 8 |
| Group 5: VLP w/ gag-flagellin + trypsin treatment | 8 |
| Group 6: VLP w/ HA-flagellin + trypsin treatment | 8 |
| Group 7: Basic VLP + trypsin treatment | 8 |

### EXAMPLE 4

### Production, Characterization and Immunogenicity Testing of gag-NA-(Norwalk-P2) VLPs

The VLP vaccine in this Example 4 will be generated in a similar vector as Example 3. The vector will express the MLV gag polypeptide and the NA polypeptide with the P2 domain of the Norwalk virus capsid protein (VP1) fused to the C-terminus of the NA polypeptide.

Initial Assay:

VLPs produced in this example will be characterized by Western blot using a Norwalk virus capsid protein-specific antibody. Since the P2 domain of the Norwalk capsid protein is the predominant antibody recognition site, Norwalk capsid-specific antibodies will be expected to recognize the P2-modified NA protein.

Size Exclusion Chromatography:

Sucrose density gradient purified VLPs will be subjected to size exclusion chromatography using Sepharose CL-4B and fractions will be monitored for MLV gag and the Norwalk P2 domain by Western blot. VLPs will elute in the void volume and will contain MLV gag, P2-modified NA.

Electron Microscopy:

VLP samples from sucrose gradients will be treated with 2% glutaraldehyde, adsorbed onto EM grids, negatively stained with sodium phosphotungstate and examined by electron microscopy.

Immunogenicity:

VLP immunogenicity will be evaluated in female Balb/c mice using intranasal chitosan/MPL formulations similar to the anthrax protective antigen (PA) formulation described in reference (44). VLPs will be purified by pelleting VLP-containing culture medium through 20% sucrose cushions at 100,000×g for 1 hour after which they will be resuspended in Tris-buffered saline and banded on 20-60% sucrose density gradients. VLP-containing fractions will be identified by Western blot assay and pooled. VLPs samples will be dialyzed into PBS and concentrated using centrifuge microconcentrators or by centrifugation at 100,000×g.

For immunization, liquid formulations (15 μl) containing 40 μg chitosan, 20 g VLP (based on gag), and 5 μg MPL will be divided between the two nostrils for a single immunization. Animals will be lightly anesthetized with isoflurane prior to intranasal dosing at 0 and 4 weeks. VLPs will also be formulated in PBS with MPL or cholera toxin for intraperitoneal inoculation as a positive control. Additional positive control animals will receive intramuscular inoculations with Systemic IgG responses specific for Norwalk P2 will be monitored by ELISA. For ELISA, the antigen source will be baculovirus-produced Norwalk VP1 capsid protein. Broncoaveolar lavage samples will also be collected 10-14 days following the final immunization for measurement of Norwalk PA-specific IgA responses by ELISA.

### EXAMPLE 5

### Production and Immunogenicity Testing of Enhanced VLPs

This Example 5 will demonstrate the enhancements of the VLPs for improved immunogenicity and protection by incorporation of the TLR5 agonist flagellin to boost the strength of adaptive immune responses.

Adjuvant Effects Due to Flagellin Incorporation:

The flagellin coding sequence was recently cloned from S. typimurium genomic DNA and inserted at the 3′ end of the gag coding sequence just 5′ to the termination codon. The flagellin coding sequence will also be inserted at the N-terminus of the A/PR/8/34 HA coding sequence using a PstI site located at the boundary between the signal peptide and mature coding sequences. Insertions at this location in HA lead to proper expression of chimeric HA molecules with expected molecular weight increases as demonstrated by SDS PAGE. The NA polypeptide will have P2 domain of the Norwalk virus (aa 279-405) fused to its C-terminus. Flagellin-modified gag and HA coding sequences will be used to generate triple baculovirus recombinants (HA-gag-NA(P2)) as described in Example 5. Recombinant baculoviruses encoding VLPs with flagellin-modified gag or flagellin-modified HA will be produced and used to generate VLPs for immunogenicity testing versus basic VLPs lacking flagellin sequences. (All VLPs will contain the Norwalk P2-modified NA as the target epitope and will either contain or lack flagellin sequences attached to gag or HA. As stated in Examples above, all immunization experiments will employ primary and booster inoculations spaced four weeks apart. Immunological readouts will be via ELISA assays as described above, examining both systemic IgG and mucosal IgA responses.

Because the HA and gag insertion sites for flagellin incorporation are outside and inside the VLP, respectively, different degrees of adjuvant effects will be observed. Flagellin insertion at the N-terminus of HA will result in easy access of flagellin to TLR5 receptors on cells in the epithelial mucosa. In contrast, the gag site of insertion will result in different access. VLP binding to cells and internalization via the normal influenza virus entry pathway will result in the deposition of the gag-flagellin product within the cell. This will result in differential TLR5-mediated adjuvant effects between the gag-flagellin and the HA-flagellin constructs. Since the ability of VLPs to bind to and enter mucosal epithelial cells may in itself have an effect on immunogenicity, we will perform VLP immunogenicity studies of flagellin-modified and normal VLPs with and without TPCK-trypsin treatment. HA cleavage of trypsin-treated VLPs will be confirmed by Western blot prior to the initiation of immunogenicity studies examining the importance of VLP entry. In addition, the ability of trypsin-treated VLPs to fuse with and enter cells will be examined by in vitro fluorescence microscopy studies employing VLPs containing a green fluorescent protein (GFP)-modified gag product. It has already been shown that MLV gag can be modified at its C-terminus with GFP without abrogation of its budding activity (60).

The use of subfragments of the flagellin coding sequence to maximize gag budding activity by eliminating much of the non-TLR5 binding regions of flagellin will also be tested. Recent mapping of the TLR5 recognition sites within the flagellin monomer will facilitate this effort (73).

TABLE 2
Example 5: Animal studies

| | # of |
|---|---|
| Flagellin-enhanced VLP Immunogenicity test | mice |
| Group 1: Neg. control | 8 |
| Group 2: VLP w/ gag-flagellin (HA-gag(flag)-NA(P2)) | 8 |
| Group 3: VLP w/ HA-flagellin (HA(flag)-gag-NA(P2)) | 8 |
| Group 4: Basic VLP (HA-gag-NA(P2)) | 8 |
| Group 5: VLP w/ gag-flagellin + trypsin treatment | 8 |
| Group 6: VLP w/ HA-flagellin + trypsin treatment | 8 |
| Group 7: Basic VLP + trypsin treatment | 8 |

### EXAMPLE 6

### Production, Characterization and Immunogenicity Testing of RSV-F-gag VLPs

The VLP vaccine for respiratory syncytial virus (RSV) in this Example will take advantage of the lipid raft targeting properties of the RSV fusion (F) protein that are similar to that of influenza HA and NA. Because of these properties, the RSV F protein it itself a lipid raft associating polypeptide and can therefore be directly incorporated into gag-based VLPs much like influenza HA and NA without the need to form chimeric proteins. To this end, the RSV F protein will be cloned by standard RT-PCR cloning techniques using the following 5′ and 3′ primers: (underlined sequences in the primers are homologous to RSV F 5′ and 3′ terminal coding sequences, while the remaining sequences contain restriction sites useful for cloning into the pFastBac1 vector).

(SEQ ID NO: 6)

5′ primer:
ATATAGGCGCGCCACCATGGAGTTGCTAATCCTCAAAGC

(SEQ ID NO: 7)

3′ primer:
ATATAGCGGCCGCTTAGTTACTAAATGCAATATTATTTATACCACTCAG

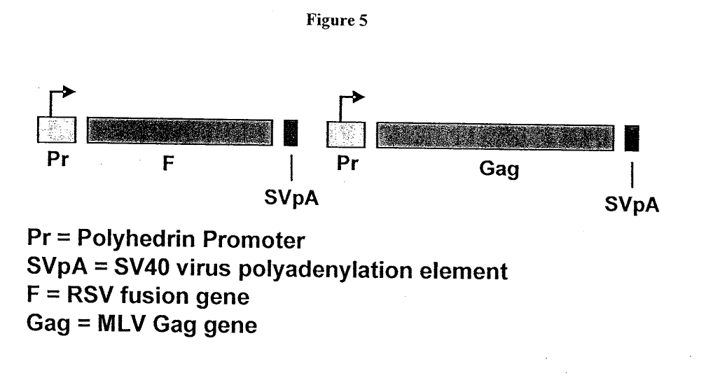

Generation of the RSV F gene by RT-PCR using the above primers will result in a fragment that can be cleaved at either end with AscI and NotI to generate cohesive ends for insertion into the pFastBac1 vector resulting in a vector called pFB-F. Upon completion of the pFB-F vector, this vector will be cleaved with HpaI for insertion of the SnaBI-HpaI fragment from pFB-gag resulting in a double baculovirus expression vector encoding both MLV-gag and RSV-F. A map of the main features of the F and Gag expression region of this plasmid is shown in FIG. 5.

When the double (F-Gag) expression vector is used to infect Sf9 cells in culture, VLPs containing the RSV F product will be observed in the culture medium because the F gene product will retain lipid raft homing sequences and will be incorporated into particles budding from lipid raft domains. Evidence for this will be shown by harvesting culture medium from cells infected with the F-Gag double expression vector, clearing the medium of debris, and collecting chimeric VLPs by centrifugation at 100,000×g over a 20% sucrose cushion. Evidence for F incorporation into the VLPs will be obtained by Western blot analysis using a specific antibody. By definition, material that sediments through a 20% sucrose cushion under these conditions is particulate in nature, providing evidence of VLP formation.

A similar approach can be used to incorporate additional RSV antigens, such as the RSV G glycoprotein.

Immunogenicity:

The immunogenicity of the RSV VLPs will be measured by the techniques used in the previous examples to measure immunogenicity of the other VLPs, except using a suitable RSV such as Human RSV stock A2 for challenge.

### ADDITIONAL REFERENCES

The following references are hereby incorporated by reference for all that they teach.
1. Katz, J. M., W. Lim, C. B. Bridges, T. Rowe, J. Hu-Primmer, X. Lu, R. A. Abernathy, M. Clarke, L. Conn, H. Kwong, M. Lee, G. Au, Y. Y. Ho, K. H. Mak, N.J. Cox, and K. Fukuda. 1999. Antibody response in individuals infected with avian influenza A (H5N1) viruses and detection of anti-H5 antibody among household and social contacts. J Infect Dis 180:1763.
2. Peiris, J. S., W. C. Yu, C. W. Leung, C. Y. Cheung, W. F. Ng, J. M. Nicholls, T. K. Ng, K. H. Chan, S. T. Lai, W. L. Lim, K. Y. Yuen, and Y. Guan. 2004. Re-emergence of fatal human influenza A subtype H5N1 disease. Lancet 363:617.
3. Horimoto, T., N. Fukuda, K. Iwatsuki-Horimoto, Y. Guan, W. Lim, M. Peiris, S. Sugii, T. Odagiri, M. Tashiro, and Y. Kawaoka. 2004. Antigenic differences between H5N1 human influenza viruses isolated in 1997 and 2003. J Vet Med Sci 66:303.
4. Tran, T. H., T. L. Nguyen, T. D. Nguyen, T. S. Luong, P. M. Pham, V. C. Nguyen, T. S. Pham, C. D. Vo, T. Q. Le, T. T. Ngo, B. K. Dao, P. P. Le, T. T. Nguyen, T. L. Hoang, V. T. Cao, T. G. Le, D. T. Nguyen, H. N. Le, K. T. Nguyen, H. S. Le, V. T. Le, D. Christiane, T. T. Tran, J. Menno de, C. Schultsz, P. Cheng, W. Lim, P. Horby, and J. Farrar. 2004. Avian influenza A (H5N1) in 10 patients in Vietnam. N Engl J Med 350:1179.
5. Li, K. S., Y. Guan, J. Wang, G. J. Smith, K. M. Xu, L. Duan, A. P. Rahardjo, P. Puthavathana, C. Buranathai, T. D. Nguyen, A. T. Estoepangestie, A. Chaisingh, P. Auewarakul, H. T. Long, N. T. Hanh, R. J. Webby, L. L. Poon, H. Chen, K. F. Shortridge, K. Y. Yuen, R. G. Webster, and J. S. Peiris. 2004. Genesis of a highly pathogenic and potentially pandemic H5N1 influenza virus in eastern Asia. Nature 430:209.
6. Lipatov, A. S., E. A. Govorkova, R. J. Webby, H. Ozaki, M. Peiris, Y. Guan, L. Poon, and R. G. Webster. 2004. Influenza: emergence and control. J Virol 78:8951.
7. Lipatov, A. S., R. J. Webby, E. A. Govorkova, S. Krauss, and R. G. Webster. 2005. Efficacy of H5 influenza vaccines produced by reverse genetics in a lethal mouse model. J Infect Dis 191:1216.
8. Stephenson, I., K. G. Nicholson, J. M. Wood, M. C. Zambon, and J. M. Katz. 2004. Confronting the avian influenza threat: vaccine development for a potential pandemic. Lancet Infect Dis 4:499.
9. Liu, M., J. M. Wood, T. Ellis, S. Krauss, P. Seiler, C. Johnson, E. Hoffmann, J. Humberd, D. Hulse, Y. Zhang, R. G. Webster, and D. R. Perez. 2003. Preparation of a standardized, efficacious agricultural H5N3 vaccine by reverse genetics. Virology 314:580.
10. Subbarao, K., H. Chen, D. Swayne, L. Mingay, E. Fodor, G. Brownlee, X. Xu, X. Lu, J. Katz, N. Cox, and Y. Matsuoka. 2003. Evaluation of a genetically modified reassortant H5N1 influenza A virus vaccine candidate generated by plasmid-based reverse genetics. Virology 305:192.
11. Webby, R. J., D. R. Perez, J. S. Coleman, Y. Guan, J. H. Knight, E. A. Govorkova, L. R. McClain-Moss, J. S. Peiris, J. E. Rehg, E. I. Tuomanen, and R. G. Webster. 2004. Responsiveness to a pandemic alert: use of reverse genetics for rapid development of influenza vaccines. Lancet 363:1099.
12. Treanor, J. J., B. E. Wilkinson, F. Masseoud, J. Hu-Primmer, R. Battaglia, D. O'Brien, M. Wolff, G. Rabinovich, W. Blackwelder, and J. M. Katz. 2001. Safety and immunogenicity of a recombinant hemagglutinin vaccine for H5 influenza in humans. Vaccine 19:1732.
13. Stephenson, I., R. Bugarini, K. G. Nicholson, A. Podda, J. M. Wood, M. C. Zambon, and J. M. Katz. 2005. Cross-reactivity to highly pathogenic avian influenza H5N1 viruses after vaccination with nonadjuvanted and MF59-adjuvanted influenza A/Duck/Singapore/97 (H5N3) vaccine: a potential priming strategy. J Infect Dis 191:1210.
14. Nicholson, K. G., A. E. Colegate, A. Podda, I. Stephenson, J. Wood, E. Ypma, and M. C. Zambon. 2001. Safety and antigenicity of non-adjuvanted and MF59-adjuvanted influenza A/Duck/Singapore/97 (H5N3) vaccine: a randomised trial of two potential vaccines against H5N1 influenza. Lancet 357:1937.
15. Subbarao, K., B. R. Murphy, and A. S. Fauci. 2006. Development of effective vaccines against pandemic influenza. Immunity 24:5.
16. Kuper, C. F., P. J. Koornstra, D. M. Hameleers, J. Biewenga, B. J. Spit, A. M. Duijvestijn, P. J. van Breda Vriesman, and T. 5 minia. 1992. The role of nasopharyngeal lymphoid tissue. Immunol Today 13:219.
17. Liang, B., L. Hyland, and S. Hou. 2001. Nasal-associated lymphoid tissue is a site of long-term virus-specific antibody production following respiratory virus infection of mice. J Virol 75:5416.
18. Zuercher, A. W., S. E. Coffin, M. C. Thurnheer, P. Fundova, and J. J. Cebra. 2002. Nasal-associated lymphoid tissue is a mucosal inductive site for virus-specific humoral and cellular immune responses. J Immunol 168:1796.
19. Brandtzaeg, P. 1989. Overview of the mucosal immune system. Curr Top Microbiol Immunol 146:13.
20. Takada, A., S. Matsushita, A. Ninomiya, Y. Kawaoka, and H. Kida. 2003. Intranasal immunization with formalin-inactivated virus vaccine induces a broad spectrum of heterosubtypic immunity against influenza A virus infection in mice. Vaccine 21:3212.
21. Tamura, S. I., H. Asanuma, Y. Ito, Y. Hirabayashi, Y. Suzuki, T. Nagamine, C. Aizawa, T. Kurata, and A. Oya. 1992. Superior cross-protective effect of nasal vaccination to subcutaneous inoculation with influenza hemagglutinin vaccine. Eur J Immunol 22:477.
22. Tumpey, T. M., M. Renshaw, J. D. Clements, and J. M. Katz. 2001. Mucosal delivery of inactivated influenza vaccine induces B-cell-dependent heterosubtypic cross-protection against lethal influenza A H5N1 virus infection. J Virol 75:5141.
23. Kang, S. M., L. Guo, Q. Yao, I. Skountzou, and R. W. Compans. 2004. Intranasal immunization with inactivated influenza virus enhances immune responses to coadministered simian-human immunodeficiency virus-like particle antigens. J Virol 78:9624.
24. Guo, L., X. Lu, S. M. Kang, C. Chen, R. W. Compans, and Q. Yao. 2003. Enhancement of mucosal immune responses by chimeric influenza HA/SHIV virus-like particles. Virology 313:502.
25. Yao, Q., R. Zhang, L. Guo, M. Li, and C. Chen. 2004. Th cell-independent immune responses to chimeric hemagglutinin/simian human immunodeficiency virus-like particles vaccine. J Immunol 173:1951.
26. Latham, T., and J. M. Galarza. 2001. Formation of wild-type and chimeric influenza virus-like particles following simultaneous expression of only four structural proteins. J Virol 75:6154.
27. Galarza, J. M., T. Latham, and A. Cupo. 2005. Virus-like particle vaccine conferred complete protection against a lethal influenza virus challenge. Viral Immunol 18:365.
28. Fromantin, C., B. Jamot, J. Cohen, L. Piroth, P. Pothier, and E. Kohli. 2001. Rotavirus 2/6 virus-like particles administered intranasally in mice, with or without the mucosal adjuvants cholera toxin and Escherichia coli heat-labile toxin, induce a Th1/Th2-like immune response. Virol 75:11010.
29. Harrington, P. R., B. Yount, R. E. Johnston, N. Davis, C. Moe, and R. S. Baric. 2002. Systemic, mucosal, and heterotypic immune induction in mice inoculated with Venezuelan equine encephalitis replicons expressing Norwalk virus-like particles. J Virol 76:730.
30. Shi, W., J. Liu, Y. Huang, and L. Qiao. 2001. Papillomavirus pseudovirus: a novel vaccine to induce mucosal and systemic cytotoxic T-lymphocyte responses. J Virol 75:10139.
31. Han, M. G., S. Cheetham, M. Azevedo, C. Thomas, and L. J. Saif. 2006. Immune responses to bovine norovirus-like particles with various adjuvants and analysis of protection in gnotobiotic calves. Vaccine 24:317.
32. Illum, L. 1998. Chitosan and its use as a pharmaceutical excipient. Pharm Res 15:1326.
33. Illum, L., I. Jabbal-Gill, M. Hinchcliffe, A. N. Fisher, and S. S. Davis. 2001. Chitosan as a novel nasal delivery system for vaccines. Adv Drug Deliv Rev 51:81.
34. Soane, R. J., M. Hinchcliffe, S. S. Davis, and L. Illum. 2001. Clearance characteristics of chitosan based formulations in the sheep nasal cavity. Int J Pharm 217:183.
35. Baudner, B. C., M. M. Giuliani, J. C. Verhoef, R. Rappuoli, H. E. Junginger, and G. D. Giudice. 2003. The concomitant use of the LTK63 mucosal adjuvant and of chitosan-based delivery system enhances the immunogenicity and efficacy of intranasally administered vaccines. Vaccine 21:3837.
36. Fujihashi, K., T. Koga, F. W. van Ginkel, Y. Hagiwara, and J. R. McGhee. 2002. A dilemma for mucosal vaccination: efficacy versus toxicity using enterotoxin-based adjuvants. Vaccine 20:2431.
37. Mutsch, M., W. Zhou, P. Rhodes, M. Bopp, R. T. Chen, T. Linder, C. Spyr, and R. Steffen. 2004. Use of the inactivated intranasal influenza vaccine and the risk of Bell's palsy in Switzerland. N Engl J Med 350:896.
38. Baldridge, J. R., Y. Yorgensen, J. R. Ward, and J. T. Ulrich. 2000. Monophosphoryl lipid A enhances mucosal and systemic immunity to vaccine antigens following intranasal administration. Vaccine 18:2416.
39. Baldrick, P., D. Richardson, G. Elliott, and A. W. Wheeler. 2002. Safety evaluation of monophosphoryl lipid A (MPL): an immunostimulatory adjuvant. Regul Toxicol Pharmacol 35:398.
40. Baldridge, J. R., P. McGowan, J. T. Evans, C. Cluff, S. Mossman, D. Johnson, and D. Persing. 2004. Taking a Toll on human disease: Toll-like receptor 4 agonists as vaccine adjuvants and monotherapeutic agents. Expert Opin Biol Ther 4:1129.
41. Baldridge, J. GlaxoSmithKline, Personal Communication.
42. Huang, J., R. J. Garmise, T. M. Crowder, K. Mar, C. R. Hwang, A. J. Hickey, J. A. Mikszta, and V. J. Sullivan. 2004. A novel dry powder influenza vaccine and intranasal delivery technology: induction of systemic and mucosal immune responses in rats. Vaccine 23:794.
43. Mills, K. H., C. Cosgrove, E. A. McNeela, A. Sexton, R. Giemza, I. Jabbal-Gill, A. Church, W. Lin, L. Illum, A. Podda, R. Rappuoli, M. Pizza, G. E. Griffin, and D. J. Lewis. 2003. Protective levels of diphtheria-neutralizing antibody induced in healthy volunteers by unilateral priming-boosting intranasal immunization associated with restricted ipsilateral mucosal secretory immunoglobulin a. Infect Immun 71:726.
44. Wimer-Mackin, S., M. Hinchcliffe, C. R. Petrie, S. J. Warwood, W. T. Tino, M. S. Williams, J. P. Stenz, A. Cheff, and C. Richardson. 2006. An intranasal vaccine targeting both the Bacillus anthracis toxin and bacterium provides protection against aerosol spore challenge in rabbits. Vaccine in press.
45. Noad, R., and P. Roy. 2003. Virus-like particles as immunogens. Trends Microbiol 11:438.
46. Yao, Q., V. Vuong, M. Li, and R. W. Compans. 2002. Intranasal immunization with SIV virus-like particles (VLPs) elicits systemic and mucosal immunity. Vaccine 20:2537.
47. Pushko, P., T. M. Tumpey, F. Bu, J. Knell, R. Robinson, and G. Smith. 2005. Influenza virus-like particles comprised of the HA, NA, and M1 proteins of H9N2 influenza virus induce protective immune responses in BALB/c mice. Vaccine 23:5751.
48. Baumert, T. F., S. Ito, D. T. Wong, and T. J. Liang. 1998. Hepatitis C virus structural proteins assemble into viruslike particles in insect cells. J Virol 72:3827.
49. Yao, Q., Z. Bu, A. Vzorov, C. Yang, and R. W. Compans. 2003. Virus-like particle and DNA-based candidate AIDS vaccines. Vaccine 21:638.
50. Tacket, C. O., M. B. Sztein, G. A. Losonsky, S. S. Wasserman, and M. K. Estes. 2003. Humoral, mucosal, and cellular immune responses to oral Norwalk virus-like particles in volunteers. Clin Immunol 108:241.
51. Gomez-Puertas, P., C. Albo, E. Pérez-Pastrana, A. Vivo, and A. Portela. 2000. Influenza virus matrix protein is the major driving force in virus budding. J Virol 74:11538.
52. Gheysen, D., E. Jacobs, F. de Foresta, C. Thiriart, M. Francotte, D. Thines, and M. De Wilde. 1989. Assembly and release of HIV-1 precursor Pr55gag virus-like particles from recombinant baculovirus-infected insect cells. Cell 59:103.
53. Johnson, M. C., H. M. Scobie, and V. M. Vogt. 2001. PR domain of rous sarcoma virus Gag causes an assembly/budding defect in insect cells. J Virol 75:4407.
54. Kakker, N. K., M. V. Mikhailov, M. V. Nermut, A. Burny, and P. Roy. 1999. Bovine leukemia virus Gag particle assembly in insect cells: formation of chimeric particles by domain-switched leukemia/lentivirus Gag polyprotein. Virology 265:308.
55. Luo, L., Y. Li, and C. Y. Kang. 1990. Expression of gag precursor protein and secretion of virus-like gag particles of HIV-2 from recombinant baculovirus-infected insect cells. Virology 179:874.
56. Morikawa, S., T. F. Booth, and D. H. Bishop. 1991. Analyses of the requirements for the synthesis of virus-like particles by feline immunodeficiency virus gag using baculovirus vectors. Virology 183:288.
57. Takahashi, R. H., K. Nagashima, T. Kurata, and H. Takahashi. 1999. Analysis of human lymphotropic T-cell virus type II-like particle production by recombinant baculovirus-infected insect cells. Virology 256:371.
58. Yamshchikov, G. V., G. D. Ritter, M. Vey, and R. W. Compans. 1995. Assembly of SIV virus-like particles containing envelope proteins using a baculovirus expression system. Virology 214:50.
59. Weldon, R. A., Jr., C. R. Erdie, M. G. Oliver, and J. W. Wills. 1990. Incorporation of chimeric gag protein into retroviral particles. J Virol 64:4169.
60. Andrawiss, M., Y. Takeuchi, L. Hewlett, and M. Collins. 2003. Murine leukemia virus particle assembly quantitated by fluorescence microscopy: role of Gag-Gag interactions and membrane association. J Virol 77:11651.
61. Leser, G. P., and R. A. Lamb. 2005. Influenza virus assembly and budding in raft-derived microdomains: a quantitative analysis of the surface distribution of HA, NA and M2 proteins. Virology 342:215.
62. Takeda, M., G. P. Leser, C. J. Russell, and R. A. Lamb. 2003. Influenza virus hemagglutinin concentrates in lipid raft microdomains for efficient viral fusion. Proc Natl Acad Sci USA 100:14610.
63. Campbell, S. M., S. M. Crowe, and J. Mak. 2001. Lipid rafts and HIV-1: from viral entry to assembly of progeny virions. J Clin Virol 22:217.
64. Sandrin, V., and F. L. Cosset. 2006. Intracellular versus cell surface assembly of retroviral pseudotypes is determined by the cellular localization of the viral glycoprotein, its capacity to interact with Gag, and the expression of the Nef protein. J Biol Chem 281:528.
65. Salazar-Gonzalez, R. M., and S. J. McSorley. 2005. Salmonella flagellin, a microbial target of the innate and adaptive immune system. Immunol Lett 101:117.
66. Cuadros, C., F. J. Lopez-Hernandez, A. L. Dominguez, M. McClelland, and J. Lustgarten. 2004. Flagellin fusion proteins as adjuvants or vaccines induce specific immune responses. Infect Immun 72:2810.
67. Didierlaurent, A., I. Ferrero, L. A. Otten, B. Dubois, M. Reinhardt, H. Carlsen, R. Blomhoff, S. Akira, J. P. Kraehenbuhl, and J. C. Sirard. 2004. Flagellin promotes myeloid differentiation factor 88-dependent development of Th2-type response. J Immunol 172:6922.
68. Tsujimoto, H., T. Uchida, P. A. Efron, P. O, Scumpia, A. Verma, T. Matsumoto, S. K. Tschoeke, R. F. Ungaro, S. Ono, S. Seki, M. J. Clare-Salzler, H. V. Baker, H. Mochizuki, R. Ramphal, and L. L. Moldawer. 2005. Flagellin enhances NK cell proliferation and activation directly and through dendritic cell-NK cell interactions. J Leukoc Biol 78:888.
69. Hayashi, F., T. K. Means, and A. D. Luster. 2003. Toll-like receptors stimulate human neutrophil function. Blood 102:2660.
70. Hayashi, F., K. D. Smith, A. Ozinsky, T. R. Hawn, E. C. Yi, D. R. Goodlett, J. K. Eng, S. Akira, D. M. Underhill, and A. Aderem. 2001. The innate immune response to bacterial flagellin is mediated by Toll-like receptor 5. Nature 410:1099.
71. Gewirtz, A. T., P. O, Simon, Jr., C. K. Schmitt, L. J. Taylor, C. H. Hagedorn, A. D. O'Brien, A. S, Neish, and J. L. Madara. 2001. Salmonella typhimurium translocates flagellin across intestinal epithelia, inducing a proinflammatory response. J Clin Invest 107:99.
72. Means, T. K., F. Hayashi, K. D. Smith, A. Aderem, and A. D. Luster. 2003. The Toll-like receptor 5 stimulus bacterial flagellin induces maturation and chemokine production in human dendritic cells. J Immunol 170:5165.
73. Smith, K. D., E. Andersen-Nissen, F. Hayashi, K. Strobe, M. A. Bergman, S. L. Barrett, B. T. Cookson, and A. Aderem. 2003. Toll-like receptor 5 recognizes a conserved site on flagellin required for protofilament formation and bacterial motility. Nat Immunol 4:1247.
74. Honko, A. N., N. Sriranganathan, C. J. Lees, and S. B. Mizel. 2006. Flagellin is an effective adjuvant for immunization against lethal respiratory challenge with Yersinia pestis. Infect Immun 74:1113.
75. Jeon, S. H., T. Ben-Yedidia, and R. Amon. 2002. Intranasal immunization with synthetic recombinant vaccine containing multiple epitopes of influenza virus. Vaccine 20:2772.
76. Levi, R., and R. Amon. 1996. Synthetic recombinant influenza vaccine induces efficient long-term immunity and cross-strain protection. Vaccine 14:85.
77. Lee, S. E., S. Y. Kim, B.C. Jeong, Y. R. Kim, S. J. Bae, O.S. Ahn, J. J. Lee, H. C. Song, J. M. Kim, H. E. Choy, S. S. Chung, M. N. Kweon, and J. H. Rhee. 2006. A bacterial flagellin, Vibrio vulnificus FlaB, has a strong mucosal adjuvant activity to induce protective immunity. Infect Immun 74:694.
78. Applequist, S. E., E. Rollman, M. D. Wareing, M. Liden, B. Rozell, J. Hinkula, and H. G. Ljunggren. 2005. Activation of innate immunity, inflammation, and potentiation of DNA vaccination through mammalian expression of the TLR5 agonist flagellin. J Immunol 175:3882.
79. Ramos, H. C., M. Rumbo, and J. C. Sirard. 2004. Bacterial flagellins: mediators of pathogenicity and host immune responses in mucosa. Trends Microbiol 12:509.
80. Holsinger, L. J., and R. A. Lamb. 1991. Influenza virus M2 integral membrane protein is a homotetramer stabilized by formation of disulfide bonds. Virology 183:32.
81. Lamb, R. A., S. L. Zebedee, and C. D. Richardson. 1985. Influenza virus M2 protein is an integral membrane protein expressed on the infected-cell surface. Cell 40:627.
82. Holsinger, L. J., D. Nichani, L. H. Pinto, and R. A. Lamb. 1994. Influenza A virus M2 ion channel protein: a structure-function analysis. J Virol 68:1551.
83. Takeda, M., A. Pekosz, K. Shuck, L. H. Pinto, and R. A. Lamb. 2002. Influenza a virus M2 ion channel activity is essential for efficient replication in tissue culture. J Virol 76:1391.
84. Frace, A. M., A. I. Klimov, T. Rowe, R. A. Black, and J. M. Katz. 1999. Modified M2 proteins produce heterotypic immunity against influenza A virus. Vaccine 17:2237.
85. Neirynck, S., T. Deroo, X. Saelens, P. Vanlandschoot, W. M. Jou, and W. Fiers. 1999. A universal influenza A vaccine based on the extracellular domain of the M2 protein. Nat Med 5:1157.
86. Slepushkin, V. A., J. M. Katz, R. A. Black, W. C. Gamble, P. A. Rota, and N.J. Cox. 1995. Protection of mice against influenza A virus challenge by vaccination with baculovirus-expressed M2 protein. Vaccine 13:1399.
87. Treanor, J. J., E. L. Tierney, S. L. Zebedee, R. A. Lamb, and B. R. Murphy. 1990. Passively transferred monoclonal antibody to the M2 protein inhibits influenza A virus replication in mice. J Virol 64:1375.
88. De Filette, M., W. Min Jou, A. Birkett, K. Lyons, B. Schultz, A. Tonkyro, S. Resch, and W. Fiers. 2005. Universal influenza A vaccine: optimization of M2-based constructs. Virology 337:149.
89. De Filette, M., A. Ramne, A. Birkett, N. Lycke, B. Lowenadler, W. Min Jou, X. Saelens, and W. Fiers. 2006. The universal influenza vaccine M2e-HBc administered intranasally in combination with the adjuvant CTA1-DD provides complete protection. Vaccine 24:544.
90. Fiers, W., M. De Filette, A. Birkett, S, Neirynck, and W. Min Jou. 2004. A “universal” human influenza A vaccine. Virus Res 103:173.
91. Liu, W., Z. Peng, Z. Liu, Y. Lu, J. Ding, and Y. H. Chen. 2004. High epitope density in a single recombinant protein molecule of the extracellular domain of influenza A virus M2 protein significantly enhances protective immunity. Vaccine 23:366.
92. Fan, J., X. Liang, M. S. Horton, H. C. Perry, M. P. Citron, G. J. Heidecker, T. M. Fu, J. Joyce, C. T. Przysiecki, P. M. Keller, V. M. Garsky, R. Ionescu, Y. Rippeon, L. Shi, M. A. Chastain, J. H. Condra, M. E. Davies, J. Liao, E. A. Emini, and J. W. Shiver. 2004. Preclinical study of influenza virus A M2 peptide conjugate vaccines in mice, ferrets, and rhesus monkeys. Vaccine 22:2993.
93. Ionescu, R. M., C. T. Przysiecki, X. Liang, V. M. Garsky, J. Fan, B. Wang, R. Troutman, Y. Rippeon, E. Flanagan, J. Shiver, and L. Shi. 2006. Pharmaceutical and immunological evaluation of human papillomavirus viruslike particle as an antigen carrier. J Pharm Sci 95:70.
94. Hatziioannou, T., E. Delahaye, F. Martin, S. J. Russell, and F. L. Cosset. 1999. Retroviral display of functional binding domains fused to the amino terminus of influenza hemagglutinin. Hum Gene Ther 10:1533.
95. Li, Z. N., S, N. Mueller, L. Ye, Z. Bu, C. Yang, R. Ahmed, and D. A. Steinhauer. 2005. Chimeric influenza virus hemagglutinin proteins containing large domains of the Bacillus anthracis protective antigen: protein characterization, incorporation into infectious influenza viruses, and antigenicity. J Virol 79:10003.
96. Haynes, J. R., S. X. Cao, B. Rovinski, C. Sia, O. James, G. A. Dekaban, and M. H. Klein. 1991. Production of immunogenic HIV-1 viruslike particles in stably engineered monkey cell lines. AIDS Res Hum Retroviruses 7:17.
97. Rovinski, B., J. R. Haynes, S. X. Cao, O. James, C. Sia, S. Zolla-Pazner, T. J. Matthews, and M. H. Klein. 1992. Expression and characterization of genetically engineered human immunodeficiency virus-like particles containing modified envelope glycoproteins: implications for development of a cross-protective AIDS vaccine. J Virol 66:4003.
98. Fynan, E. F., R. G. Webster, D. H. Fuller, J. R. Haynes, J. C. Santoro, and H. L. Robinson. 1995. DNA vaccines: a novel approach to immunization. Int J Immunopharmacol 17:79.
99. Fynan, E. F., R. G. Webster, D. H. Fuller, J. R. Haynes, J. C. Santoro, and H. L. Robinson. 1993. DNA vaccines: protective immunizations by parenteral, mucosal, and gene-gun inoculations. Proc Natl Acad Sci USA 90:11478.
100. Kodihalli, S., J. R. Haynes, H. L. Robinson, and R. G. Webster. 1997. Cross-protection among lethal H₅N₂ influenza viruses induced by DNA vaccine to the hemagglutinin. J Virol 71:3391.
101. Robinson, H. L., S. Lu, D. M. Feltquate, C. T. Torres, J. Richmond, C. M. Boyle, M. J. Morin, J. C. Santoro, R. G. Webster, D. Montefiori, Y. Yasutomi, N. L. Letvin, K. Manson, M. Wyand, and J. R. Haynes. 1996. DNA vaccines. AIDS Res Hum Retroviruses 12:455.
102. Drape, R. J., M. D. Macklin, L. J. Barr, S. Jones, J. R. Haynes, and H. J. Dean. 2006. Epidermal DNA vaccine for influenza is immunogenic in humans. Vaccine in press.
103. Kretzchmar, E., R. Geyer, and H. D. Klenk. 1994. Baculovirus infection does not alter N-glycosylation in Spodoptera frugiperda cells. Biol Chem Hoppe Seyler 375:23.
104. Lu, D., and A. J. Hickey. 2005. Liposomal dry powders as aerosols for pulmonary delivery of proteins. AAPS PharmSciTech 6:E641.
105. Cowdery, S., M. Frey, S. Orlowski, and A. Gray. 1976. Stability characteristics of freeze-dried human live virus vaccines. Dev Biol Stand 36:297.
106. Peetermans, J., G. Colinet, A. Bouillet, E. D'Hondt, and J. Stephenne. 1976. Stability of live, freeze-dried virus vaccines. Dev Biol Stand 36:291.
107. Yannarell, D. A., K. M. Goldberg, and R. N. Hjorth. 2002. Stabilizing cold-adapted influenza virus vaccine under various storage conditions. J Virol Methods 102:15.
108. Sampson, H. A., J. Bernhisel-Broadbent, E. Yang, and S. M. Scanlon. 1991. Safety of casein hydrolysate formula in children with cow milk allergy. J Pediatr 118:520.
109. Gambaryan A, A. Tuzikov, G. Pazynina, N. Bovin, A. Balish, and A. Klimov. 2005. Evolution of the receptor binding phenotype of influenza A (H5) viruses. Virology (electronic publication ahead of print version).
110. Suzuki, Y, 2005. Sialobiology of Influenza: Molecular Mechanism of Host Range Variation of Influenza Viruses. Biological and Pharmaceutical Bulletin 28:399-408.

What we claim is:

1. A chimeric virus-like particle comprising:
(a) a gag polypeptide; and
(b) a non-viral lipid raft-associated polypeptide.

2. The virus-like particle of claim 1, wherein the lipid raft-associated polypeptide is selected from the group consisting of a GPI anchor polypeptide, a myristoylation sequence polypeptide, a palmitoylation sequence polypeptide, a double acetylation sequence polypeptide, a signal transduction polypeptide, and a membrane trafficking polypeptide.

3. The virus-like particle of claim 1, wherein the lipid raft-associated polypeptide is selected from the group consisting of a GPI anchor polypeptide, a myristoylation sequence polypeptide, a palmitoylation sequence polypeptide, a double acetylation sequence polypeptide, a cavelin polypeptide, a flotillin polypeptide, a syntaxin-1 polypeptide, a syntaxin-4 polypeptide, a synapsin I polypeptide, an adducin polypeptide, a VAMP2 polypeptide, a VAMP/synaptobrevin polypeptide, a synaptobrevin II polypeptide, a SNARE polypeptide, a SNAP-25 polypeptide, a SNAP-23 polypeptide, a synaptotagmin I polypeptide, and a synaptotagmin II polypeptide.

4. The virus-like particle of claim 1 further comprising a hemagglutinin polypeptide.

5. The virus-like particle of claim 4 further comprising a neuraminidase polypeptide.

6. A chimeric virus-like particle comprising:
(a) a gag polypeptide; and
(b) a lipid raft-associated polypeptide linked to an antigen to form a linkage, wherein said antigen is not naturally associated with a lipid raft.

7. The virus-like particle of claim 6, wherein said linkage is selected from the group consisting of: a covalent bond, an ionic interaction, a hydrogen bond, an ionic bond, a van der Waals force, a metal-ligand interaction, and an antibody-antigen interaction.

8. The virus-like particle of claim 6, wherein said linkage is a covalent bond.

9. The virus-like particle of claim 8, wherein said covalent bond is selected from the group consisting of: a peptide bond, a carbon-oxygen bond, a carbon-sulfur bond, a carbon-nitrogen bond, a carbon-carbon bond, and a disulfide bond.

10. The virus-like particle of claim 6, wherein said lipid raft-associated polypeptide is an integral membrane protein.

11. The virus-like particle of claim 6, wherein said lipid raft-associated polypeptide is selected from the group consisting of: a hemagglutinin polypeptide, a neuraminidase polypeptide, a fusion protein polypeptide, a glycoprotein polypeptide, and an envelope protein polypeptide.

12. The virus-like particle of claim 11, wherein said lipid raft-associated polypeptide is a hemagglutinin polypeptide.

13. The virus-like particle of claim 12 further comprising a neuraminidase polypeptide.

14. The virus-like particle of claim 11, wherein said lipid raft-associated polypeptide is a neuraminidase polypeptide.

15. The virus-like particle of claim 14 further comprising a hemagglutinin polypeptide.

16. The virus-like particle of claim 6, wherein said antigen is selected from the group consisting of: a protein, a polypeptide, a glycopolypeptide, a lipopolypeptide, a peptide, a polysaccharide, a polysaccharide conjugate, a peptide or non-peptide mimic of a polysaccharide, a small molecule, a lipid, a glycolipid, and a carbohydrate.

17. The virus-like particle of claim 6 wherein the virus-like particle comprises insect cell glycosylation.

18. The virus-like particle of claim 6 wherein the virus-like particle comprises mammalian cell glycosylation.

19. The virus-like particle of claim 6 further comprising a second lipid-raft associated polypeptide.

20. The virus-like particle of claim 6, further comprising an adjuvant in admixture with said virus-like particle.

21. The virus-like particle of claim 20, wherein said adjuvant is located inside said virus-like particle.

22. The virus-like particle of claim 21, wherein said adjuvant is covalently linked to said gag polypeptide to form a covalent linkage.

23. The virus-like particle of claim 20, wherein said adjuvant is located outside said virus-like particle.

24. The virus-like particle of claim 23, wherein said adjuvant is covalently linked to said lipid raft-associated polypeptide to form a covalent linkage.

25. The virus-like particle of claim 20, wherein said adjuvant comprises an adjuvant-active fragment of flagellin.

26. A chimeric virus-like particle comprising:
(a) a gag polypeptide; and
(b) an RSV lipid raft-associated polypeptide.

27. The virus-like particle of claim 26 wherein the virus-like particle comprises insect cell glycosylation.

28. The virus-like particle of claim 26 wherein the virus-like particle comprises mammalian cell glycosylation.

29. The virus-like particle of claim 26 further comprising a second lipid-raft associated polypeptide.

30. The virus-like particle of claim 26 further comprising an antigen linked to the RSV lipid raft-associated polypeptide to form a linkage.

31. The virus-like particle of claim 29 wherein the second lipid-raft associated polypeptide is or is linked to a second RSV antigen.

32. The virus-like particle of claim 29 wherein the second lipid-raft associated polypeptide is linked to an RSV antigen.

33. The virus-like particle of claim 26, further comprising an adjuvant in admixture with said virus-like particle.

34. A chimeric virus-like particle comprising:
(a) a gag polypeptide; and
(b) an enveloped virus lipid raft-associated polypeptide;
wherein the virus-like particle comprises insect cell glycosylation.

35. The virus-like particle of claim 34 wherein the enveloped virus lipid raft-associated polypeptide is selected from the group consisting of a Paramyxoviridae and Herpesviridae lipid raft-associated polypeptide.

36. The virus-like particle of claim 35 wherein the lipid raft-associated polypeptide is selected from the group consisting of parainfluenza virus, mumps virus, measles virus, respiratory syncytial virus, Avian pneumovirus, Human metapneumovirus, herpes simplex virus 1 (HHV-1), herpes simplex virus 2(HHV-2), varicella zoster virus (HHV-3), Epstein Barr virus (HHV-4), and cytomegalovirus (CMV) (HHV-5) lipid raft-associated polypeptides.

37. The virus-like particle of claim 34 further comprising a second lipid-raft associated polypeptide.

38. The virus-like particle of claim 34, further comprising an adjuvant in admixture with said virus-like particle.

39. A chimeric virus-like particle expression vector system, which comprises:
(a) a first nucleotide sequence encoding a gag polypeptide; and
(b) a second nucleotide sequence encoding a lipid raft-associated polypeptide linked to an antigen, wherein said antigen is not naturally associated with a lipid raft,
wherein upon expression in a cellular host, said polypeptides form a virus-like particle.

40. The virus-like particle expression vector system of claim 39, wherein said first and second nucleotide sequences are in a single expression vector.

41. The virus-like particle expression vector system of claim 40, wherein said first and second nucleotide sequences are operably linked to a single promoter.

42. The virus-like particle expression vector system of claim 39, wherein said first and second nucleotide sequences are in multiple expression vectors.

43. A method for producing a chimeric virus-like particle, comprising:
(a) providing one or more expression vectors, together which express a gag polypeptide and a lipid raft-associated polypeptide linked to an antigen, wherein said antigen is not naturally associated with a lipid raft;
(b) introducing said one or more expression vectors into a cell; and
(c) expressing said gag polypeptide and said lipid raft-associated polypeptide linked to an antigen to produce said virus-like particle.

44. The method of claim 43, further comprising the step of recovering said virus-like particle from the media in which said cell is cultured.

45. The method of claim 43, wherein said one or more expression vectors is a viral vector.

46. The method of claim 45, wherein said viral vector is selected from the group consisting of: a baculovirus, an adenovirus, a herpesvirus, a poxvirus and a retrovirus.

47. The method of claim 46, wherein said viral vector is a baculovirus.

48. The method of claim 43, wherein said cell is selected from the group consisting of: an insect cell and a mammalian cell.

49. The method of claim 48, wherein said cell is an insect cell.

50. The method of claim 43, wherein said one or more expression vectors is a baculovirus and said cell is an insect cell.

51. The method of claim 43, further comprising the step of recovering said virus-like particle from the media in which said cell is cultured.

52. A method for treating or preventing a disease or symptom of the immune system, comprising administering an immunogenic amount of a chimeric virus-like particle, wherein said particle comprises a gag polypeptide and a lipid raft-associated polypeptide linked to an antigen, wherein said antigen is not naturally associated with a lipid raft.

53. The method of claim 52, wherein the administering induces a protective immunization response in the subject.

54. The method of claim 52, wherein the administering is selected from the group consisting of subcutaneous delivery, transcutaneous delivery, intradermal delivery, subdermal delivery, intramuscularly delivery, peroral delivery, oral delivery, intranasal delivery, buccal delivery, sublinqual delivery, intraperitoneal delivery, intravaginal delivery, anal delivery and intracranial delivery.
